SEQUENCE LISTING (1 sequence)

SEQ ID NO: 1 - protein, 354 aa, Homo sapiens
MKSLLLLVLISICWADHLSDNYTLDHDRAIHIQAENGPHLLVEAEQAKVFSHRGGNVTLPCKFYRDPTAFGSGIHKIRIKWTKLTSDYLKEVDVFVSMGYHKKTYGGYQGRVFLKGGSDSDASLVITDLTLEDYGRYKCEVIEGLEDDTVVVALDLQGVVFPYFPRLGRYNLNFHEAQQACLDQDAVIASFDQLYDAWRGGLDWCNAGWLSDGSVQYPITKPREPCGGQNTVPGVRNYGFWDKDKSRYDVFCFTSNFNGRFYYLIHPTKLTYDEAVQACLNDGAQIAKVGQIFAAWKILGYDRCDAGWLADGSVRYPISRPRRRCSPTEAAVRFVGFPDKKHKLYGVYCFRAYN

(19) United States
(12) Patent Application Publication
(10) Pub. No.: US 2025/0109384 A1
(43) Pub. Date: Apr. 3, 2025

(54) A CELLULAR COMPOSITE

(71) Applicant: ALCYOMICS LIMITED, Newcastle upon Tyne (GB)

(72) Inventors: Anne DICKINSON, Newcastle upon Tyne (GB); Mahid AHMED, Newcastle upon Tyne (GB)

(21) Appl. No.: 18/728,390

(22) PCT Filed: Jan. 13, 2023
(86) PCT No.: PCT/GB2023/050064

§ 371 Date: Jul. 11, 2024

(30) Foreign Application Priority Data
Jan. 13, 2022 (GB) 2200419.6

(51) Int. Cl. C12N 5/077

(52) U.S. Cl. CPC C12N 5/0655; C12N 2501/20; C12N 2501/2301; C12N 2502/1311; C12N 2503/02; C12N 2506/1346; C12N 2513/00; C12N 2533/80

(57) ABSTRACT

The present invention relates to a cellular composite comprising a 3D (three dimensional) cell growth material within which a population of chondrocytes is distributed, and which has a surface that is coated with a population of osteoblasts. The invention also relates to a method of producing said cellular composite and composites produced by the method of the invention. Further the invention relates to an in vitro model for studying healthy or diseased articular cartilage, as well as uses of the composite as an in vitro model. Finally, the invention relates to a method of screening an agent for the treatment or prevention of articular cartilage disease.

### FIELD OF INVENTION

The present invention relates to a cellular composite comprising a 3D (three dimensional) cell growth material within which a population of chondrocytes is distributed, and which has a surface that is coated with a population of osteoblasts. The invention also relates to a method of producing said cellular composite and composites produced by the method of the invention. Further the invention relates to an in vitro model for studying healthy or diseased articular cartilage, as well as uses of the composite as an in vitro model. Finally, the invention relates to a method of screening an agent for the treatment or prevention of articular cartilage disease.

### BACKGROUND

Osteoarthritis (OA) is a chronic degenerative disease of the articular joint that involves cartilage, bone, synovium, tendons and ligaments. Articular cartilage is a highly specialised connective tissue of avascular lining chondrocytes that support load transfer and motion. The cartilage has a low cellularity and low cell turnover which makes natural regeneration process a major challenge. This damage can be caused by various kinds of pathologies such as osteochondritis, osteonecrosis and haemoarthrosis. Prolonged inflammatory processes results in progressive degeneration of the extracellular matrix (ECM) in the cartilage and subsequent deterioration of subchondral bone due to osteoclast activation. Small localised defects are normally healed by compensation of ECM generation by chondrocytes in the cartilage, however; this balance is compromised during chronic diseases. Currently, palliative treatment is the main approach to relieve the symptoms of OA in absence of effective therapy that keeps and maintains the integrity of the cartilage. There is also a surgical procedure which mostly depends on a replacement approach and has a variety of restrictions and complications.

Mesenchymal stem cells (MSCs) are pluripotent cells that can give rise to skeletal tissues such as marrow stroma, bones, ligaments and cartilages. These cells have the potential of being a highly valuable source in regenerative medicine and for cartilage repair. However, previous experience showed that chondrogenic differentiation of MSCs in 3D hyaluronic acid hydrogels had challenges to recapitulate the functional properties of native articular cartilage when compared to donated articular chondrocytes. MSCs, upon chondrogenic induction, showed a hypertrophic phenotype resulting in undesired calcification of the extracellular matrix (ECM) after ectopic transplantation.

Articular joint models were created and developed in order to compensate the absence of practical easy-to-use animal models. Tissue engineered models can be used for drug screening and toxicity assessment, as well as in vitro pre-clinical models of normal tissue function including innervation. These models used different approaches in generating chondrocytes from mesenchymal stromal cells following chondrogenesis in 3D media. Early 3D models were initiated with in vitro techniques of limb bud mesenchymal cells undergoing chondrogenic differentiation. Cells were condensed and aggregated through differentiation into cartilage nodules; this system has been used as a platform for drug testing. However this model uses only one cell type, which is not reflective of in vivo conditions. Another approach used micromass pellets in co-culturing of human articular chondrocytes (HAC) with MSCs in vitro. Cell pellets of high-density droplets of MSCs was another way of 3D model development for chondrogenesis following mild centrifugation. However, clinical usage of these pellets resulted in short-term cartilage formation and calcification as a sign of hypertrophy. The third approach is the biomaterial scaffold where cells are seeded in a 3D membrane in the presence of chondrogenic media. These structures may include hydrogels, sponges and fibrils made from natural and synthetic materials that support chondrogenic differentiation. This structure supports chondrogenesis in contexts such as in vitro differentiation and repair of articular cartilage. However, these structures also do not reflect in vivo conditions very well because there is less ECM and they have reduced ability to mesh with the cells. Plugs of diseased tissue is another way of studying OA, but harvesting plugs can be invasive.

The present invention aims to address some of the problems in the prior art and provide a new and improved model of articular cartilage which would be particularly useful in the context of in vitro screening and drug development.

### SUMMARY OF INVENTION

In one aspect, the invention provides a cellular composite comprising a 3D (three dimensional) cell growth material within which a population of chondrocytes is distributed, and which has a surface that is coated with a population of osteoblasts.

Suitably, the population of chondrocytes may have normal expression levels of a protein selected from the group consisting of aggrecan (ACAN), SOX9, ADAMTS5, MMP9 and MMP13.

Suitably, the normal expression levels may be substantially the same as expression levels in healthy chondrocytes.

Suitably, the cellular composite may comprise aggrecan protein.

Suitably, the aggrecan protein may be produced by the population of chondrocytes.

Suitably, the population of chondrocytes may have abnormal expression levels of a protein selected from the group consisting of ACAN, SOX9, ADAMTS5, MMP9 and MMP13.

Suitably, the abnormal expression levels may be substantially the same as expression levels in chondrocytes from a subject having osteoarthritis and/or expression levels in chondrocytes that may have been exposed to osteoarthritic inducing cytokines, optionally the cytokines maybe selected from the group consisting of IL1-α and/or oncostatin M.

Suitably, the chondrocytes and/or osteoblasts may be derived from mesenchymal stem cells (MSCs).

Suitably, the MSCs may be Y201 cells.

Suitably, the chondrocytes and/or osteoblasts may be human cells.

Suitably, the 3D cell growth material is formed from a porous scaffold or a gel.

Suitably, the pores may be between about 25-500 μm, or between about 100-300 μm, or between about 150-250 μm.

Suitably, the scaffold may comprise or consist of a polymer, optionally wherein the polymer may be selected from the group consisting of polystyrene, Teflon®, polycarbonate, polyester, or acrylate, further optionally wherein the scaffold may be Alvetex®.

Suitably, the gel may be a hydrogel, optionally wherein the hydrogel is selected from the group consisting of HydroMatrix™ Peptide Hydrogel, MaxGel™ Human ECM, Hystem® Stem Cell Culture, Geltrex®, or Matrigel™.

Suitably, the composite may comprise a further population of cells, optionally wherein the further population of cells may comprise or consist of neurons and/or Schwann cells.

In one aspect, the invention provides a method of producing a cellular composite comprising:
a) distributing a population of chondrocytes within a 3D cell growth material; and
   b) coating a surface of the 3D cell growth material with a population of osteoblasts.

Suitably, the step of distributing a population of chondrocytes may comprise seeding a population of mesenchymal stem cells (MSCs) within the 3D cell growth material and differentiating the MSCs into chondrocytes.

Suitably the population of chondrocytes has been or may be differentiated from MSCs in a cell culture medium supplemented with a protein of the hyaluronan and proteoglycan binding link protein (HAPLN) family (for example HAPLN1, optionally wherein the HAPLN1 protein has a sequence that is at least 80% identical to SEQ ID NO: 1).

In a related aspect, the invention provides a method of producing a cellular composite comprising:
a) distributing a population of mesenchymal stem cells (MSCs) within a 3D cell growth material and differentiating the MSCs into chondrocytes in a cell culture medium supplemented with a protein of the hyaluronan and proteoglycan binding link protein (HAPLN) family; and
b) coating a surface of the 3D cell growth material with a population of osteoblasts.

Suitably, in the methods of the invention, the surface of the 3D cell growth material is coated with a population of osteoblasts after MSCs have been differentiated into chondrocytes.

In a related aspect, the invention provides a method of producing a cellular composite comprising:
a) distributing a population of chondrocytes differentiated in a cell culture medium supplemented with a protein of the hyaluronan and proteoglycan binding link protein (HAPLN) family within a 3D cell growth material; and
b) coating a surface of the 3D cell growth material with a population of osteoblasts.

Suitably, the method may further comprise the step of differentiating MSCs into chondrocytes in a cell culture medium supplemented with a protein of the HAPLN family prior to step a).

Suitably, in the methods of the invention, the protein of the HAPLN family may be HAPLN1.

Suitably, in the methods of the invention, HAPLN1 may have a sequence that is at least 80% identical to SEQ ID NO: 1.

Suitably, in the methods of the invention, the cell culture medium may be supplemented with a protein of the HAPLN family at a concentration of about 50 ng/ml to about 150 ng/ml, optionally about 100 ng/ml.

Suitably, in the methods of the invention, the cell culture medium may be supplemented with the protein of the HAPLN family (such as HAPLN1) from day 0 of the differentiation step.

Suitably, in the methods of the invention, the cell culture medium may be supplemented with the protein of the HAPLN family (such as HAPLN1) from about day 0 to about day 28 of the differentiation step.

Suitably, the methods of the invention may further comprise contacting the population of chondrocytes with IL1-α and/or oncostatin M after step b).

Suitably, the population of chondrocytes may be contacted with IL1-α and/or oncostatin M for about 1 day, 2 days, about 3 days, about 4 days, about 5 days, about 6 days, about 7 days, about 8 days, about 9 days, or about 10 days.

Suitably, in the methods of the invention, the chondrocytes and/or osteoblasts may be derived from MSCs.

Suitably, in the methods of the invention, the MSCs may be immortalised (for example Y201 cells) or primary.

Suitably, in the methods of the invention, the cells may be human cells.

Suitably, in the methods of the invention, the 3D cell growth material may be inert.

Suitably, in the methods of the invention, the 3D cell growth material may be formed from a porous scaffold and/or a gel.

Suitably, in the methods of the invention, the scaffold may comprise or consist of a polymer, optionally wherein the polymer may be selected from the group consisting of polystyrene, Teflon®, polycarbonate, polyester, or acrylate, further optionally wherein the scaffold may be Alvetex®.

Suitably, in the methods of the invention, the gel may be a hydrogel, optionally wherein the hydrogel may be selected from the group consisting of HydroMatrix™ Peptide Hydrogel, MaxGel™ Human ECM, Hystem® Stem Cell Culture, Geltrex®, or Matrigel™.

Suitably, in the methods of the invention, the pores may be between about 25-500 μm, or between about 100-300 μm, or between about 150-250 μm.

Suitably, the methods of the invention may further comprise providing a further population of cells, optionally wherein the further population of cells comprises or consists of neurons and/or Schwann cells. Suitably, the population of neurons and/or Schwann cells may be provided by distributing within the 3D cell growth material.

In one aspect, the invention provides a cellular composite produced by a method of the invention.

In one aspect, the invention provides an in vitro model for studying healthy or diseased articular cartilage comprising the composite of the invention.

Suitably, the disease may be selected from the group consisting of osteoarthritis, osteoarthrosis and rheumatoid arthritis.

In one aspect, the invention provides use of a cellular composite of the invention an in vitro model of articular cartilage.

Suitably, the articular cartilage may be heathy or diseased.

Suitably, the disease may be selected from the group consisting of osteoarthritis, osteoarthrosis and rheumatoid arthritis.

In one aspect, the invention provides a method of screening an agent for the treatment and/or prevention of articular cartilage disease, comprising:
a) providing a cellular composite of the invention;
b) exposing the cellular composite to the agent; and
c) determining whether the agent has a therapeutic effect on the composite.

Suitably, the articular cartilage disease may be selected from the group consisting of osteoarthritis, osteoarthrosis and rheumatoid arthritis.

Suitably, when the agent is for treatment, the population of chondrocytes may have abnormal expression levels of a protein selected from the group consisting of aggrecan, SOX9, ADAMTS5, MMP9 and MMP13.

Suitably, when the agent is for prevention, the population of chondrocytes may have normal expression levels of a protein selected from the group consisting of aggrecan, SOX9, ADAMTS5, MMP9 and MMP13.

It will be appreciated that except for where the context requires otherwise, embodiments described with reference to one aspect of the invention may also be applied to other aspects of the invention. For example any embodiment provided in relation to the cellular composite of the invention also applies to any of the methods of the invention, unless the context requires otherwise.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE DRAWINGS

Embodiments of the invention are further described hereinafter with reference to the accompanying drawings, in which:

FIG. 1 depicts chondrocytes in 3D culture. A) pellet of MSCs differentiated in chondrogenesis media and stained in H&E. B) Alvetex membrane in which MSCs are differentiated to chondrocytes and stained in H&E. C) pellet of MSCs differentiated in chondrogenesis media and stained in Alcian blue. D) Alvetex membrane in which MSCs are differentiated to chondrocytes and stained.

FIG. 2 depicts the two-layer model (FFPE), Chondrocytes-Osteoblast stained by H&E. Y201 cells were differentiated in Alvetex membrane (28 days) in chondrogenic media, Y201-derived osteoblasts were seeded on top of them. The two-layer model was immersed in DMEM media.

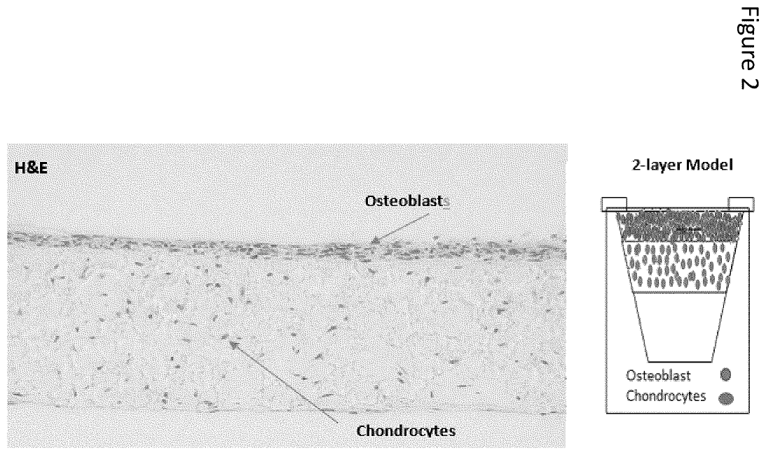

FIG. 3 depicts Alcian blue staining of 3D chondrocyte-osteoblast model. A) chondrogenesis in 3D pellet, B) and C) chondrogenesis in 3D Alvetex membrane two-layer model stained by Alcian Blue.

FIG. 4 depicts immunofluorescence staining with anti-aggrecan, Collagen2A1 and SOX9 antibodies. A, E and I) represent no primary antibody; B, F and J represent chondrogenesis in 3D sphere pellet. C, G and K) represent chondrogenesis in 3D Alvetex membrane; D, H and L) represent two-layer model of chondrocytes and osteoblasts.

FIG. 5 depicts gene expression in two-layer model following chondrogenesis for 28 days. A) shows negative values of ΔΔCT of SOX9 referenced to Y201 and housekeeping genes (GAPDH). B) shows values of ΔΔCT of aggrecan and SOX9 for 3D Alvetex and sphere pellets referenced to TC28A2 cell line. Error bars represent the mean of error.

FIG. 6 depicts FFPE sections of the two-layer model with induced OA and stained with Alcian blue. Two-layer models were incubated in DMEM (A, B and C) or osteochondral media (D, E and F) for three days then with two concentrations C1 (B and E) and C2 (C and F) for six days. A and D were incubated in media only and no treatment as controls. “C1” as used herein is IL1-α at 50 ng/ml and oncostatin M at 10 ng/m. “C2” as used herein is IL1-α at 100 ng/ml and oncostatin M at 20 ng/m.

FIG. 7 depicts immunofluorescence staining with anti-SOX9 antibodies. A, E) represent no primary antibody; B and F) represent models with no treatment (media only) whereas C, D and G, H represent two concentrations of added cytokines at C1 (C and G) and C2 (D and H) respectively. The upper images (A to D) were incubated in DMEM whereas the lower images (E to H) were incubated in osteochondral (OC) media. Reduced SOX9 expression can be seen where the two-layer model was incubated with anti SOX9 antibody.

FIG. 8 depicts gene expression in two-layer model following induction of OA at two concentrations C1, C2 and in two media; DMEM (A) and OC media (B). The results show decreased levels of aggrecan. RNA was isolated from three biological replicates and investigated by qPCR (TaqMan assay). Data was presented as fold change in referenced to no treatment control and GAPDH housekeeping gene. Error bars represent the mean of error.

FIG. 9 depicts gene expression in two-layer model following induction of OA at two concentrations C1, C2 and in two media DMEM and OC media (A and B respectively). The results show increased levels of MMP9, MMP13, and ADAMTS5. RNA was isolated from three biological replicates and investigated by qPCR (TaqMan assay). Data was presented as fold change in reference to no treatment control and GAPDH housekeeping gene. Error bars represent the mean of error.

FIG. 10 depicts gene expression in two-layer model following induction of OA at two concentrations C1, C2 and in OC media only, one hand made (A) and one bioprinted (B). Runt-related transcription factor 2 (Runx2) is a key transcription factor controlling osteoblast and chondrocyte differentiation and is upregulated in osteoarthritis and alkaline phosphatase (ALP) which is also increased in osteoarthritis. RNA was isolated from three biological replicates and investigated by qPCR (TaqMan assay). Data was presented as fold change in reference to no treatment control and GAPDH housekeeping genes. Error bars represent the mean of error. The bioprinted model showed convincing increased levels of both genes.

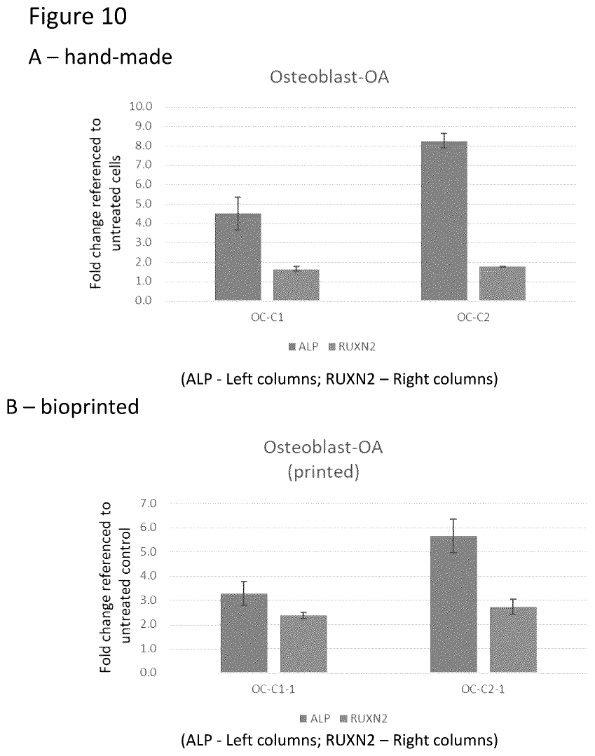

FIG. 11 depicts the results of an assay measuring gene expression levels of ACAN, SOX9, ADAMS5, MMP9 and MMP13. Increased levels of ADAMS5, MMP9 and MMP13 especially in osteochondral media indicate osteogenic type of damage induction.

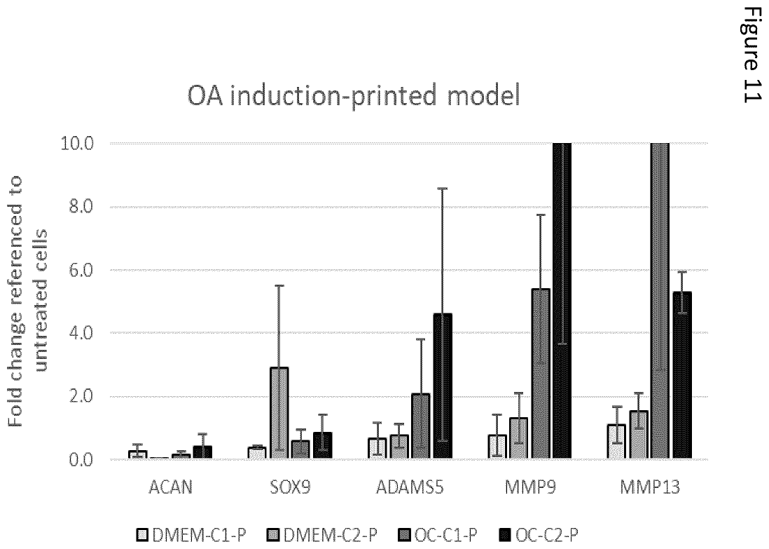

FIG. 12 depicts the OA model with depleted levels of aggrecan in the cytokine treated model demonstrating damage induced by cytokines IL1-α and oncostatin M.

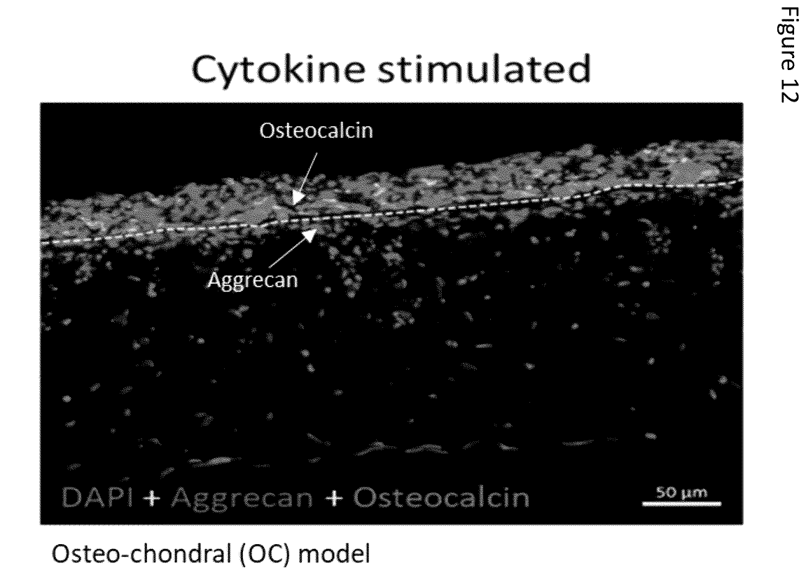

FIG. 13 depicts MMP activity of Y201 derived OA models treated with cytokines and a small molecule consisting of a mixture of anti-ADAMTS-4/ADAMTS-5, and MMP Inhibitor as determined via FRET assay.

FIG. 14 depicts a volcano plot of RNA sequencing analysis of the OA models. Data is presented as a fold change in gene expression of cytokine stimulated models compared to unstimulated models.

FIG. 15 depicts phenotypic analysis of primary cells and Y201 MSCs. A-D) Cells stained for a cocktail of negative MSC markers. E-H) Cells stained for CD90. I-L) Cells stained for CD105. M-P) Cells stained for CD73.

FIG. 16 depicts gene expression analysis of OA models with primary cells via q-PCR. Data is presented as fold change of gene expression in cytokine stimulated models (OA) in relation to untreated controls. A) Donor 3037B. B) Donor 1936B, C) Donor 1932B.

FIG. 17 depicts ELISA analysis of supernatants from primary MSC based models versus OA models. A) Quatification of MMP13. B) Quantification of ADAMTS4. N=3

FIG. 18 depicts MMP activity as determined via FRET assay. N=3

FIG. 19 depicts gene expression analysis of OA models. A) uncoated Alvetex. B) Hyaluronan coated Alvetex C) Collagen II coated Alvetex D) HAPLN1 modified chondrogenic media E) aggrecan coated Alvetex. N=3.

FIG. 20 depicts representative images of aggrecan immunofluorescence staining for models and OA models modified with coatings or chondrogenic media modification. Aggrecan is observable as a matrix. Cell nuclei are observable as circular entities.

FIG. 21 depicts MMP13 concentrations quantified via ELISA of models and OA models. A) Without coating B) Collagen II coated scaffolds C) Hyaluronan coated scaffolds D) HAPLN1 supplemented scaffolds E) Aggrecan coated scaffolds. N=3.

FIG. 22 depicts ADAMTS4 concentrations quantified via ELISA of models and OA models. A) Without coating B) Collagen II coated scaffolds C) Hyaluronan coated scaffolds D) HAPLN1 supplemented scaffolds E) Aggrecan coated scaffolds. N=3.

FIG. 23 depicts MMP activity as determined by FRET assay.

FIG. 24 depicts immunofluorescence staining of aggrecan. It can be seen from panel A that a cellular composite comprising chondrocytes that have not been differentiated in the presence of HAPLN1 protein, has almost undetectable amounts of ACAN. That is despite normal ACAN expression levels. From panel B it can be seen that the cellular composite comprising chondrocytes that have been differentiated from MSCs in the presence of HAPLN1 has significant levels of ACAN protein (indicated with brackets). Panel C shows that upon exposure to the cytokines, there is a partial loss of ACAN in the ECM.

FIG. 25 depicts immunofluorescence staining of collagen II. This figure confirms that differentiation of MSCs to chondrocytes in the presence of HAPLN1 protein does not have a negative effect on collagen II production. Collagen II highlighted with brackets. Upon stimulation with cytokines however, a reduction in the amount of collagen II was observed.

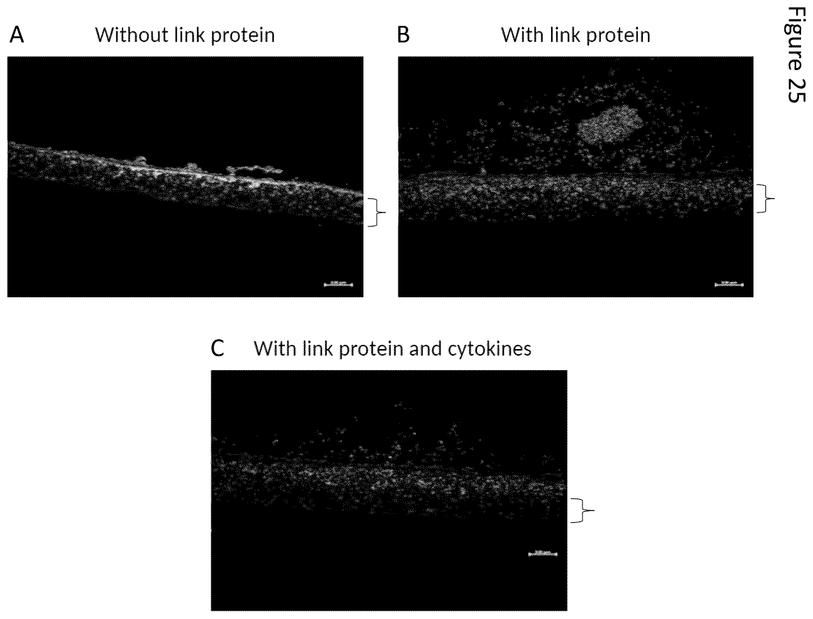

FIG. 26 illustrates a cellular composite in which the osteoblasts were cultured in a 3D scaffold and were coated with a layer of chondrocytes. 10 days after co-culture chondrocytes gradually died and detached from the layer.

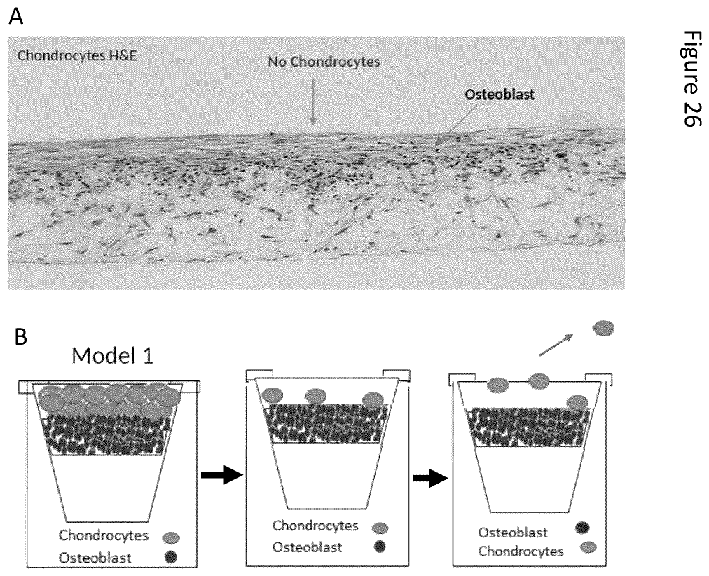

### DETAILED DESCRIPTION

The present invention is based upon the inventors' development of a cellular construct that can be used to model healthy or diseased cartilage. Upon extensive experimentation and specific selection of a variety of parameters, the inventors have developed a construct that can model both healthy or diseased cartilage as evidenced by gene expression studies of genes such as ACAN, SOX9, ADAMTS5, MMP9 and MMP13. Contrary to some of the models in the prior art, the construct is simple to produce and use. The cellular construct of the invention provides a much needed tool for in vitro research. In particular it can be very useful for screening potential therapeutic agents as novel therapies for diseases of the cartilage, such as osteoarthritis. It may also be a useful tool for determining if an individual will benefit from a therapeutic agent, as not all individuals might respond to known therapeutic agents in the same manner.

The cellular construct described herein is advantageous because it comprises aggrecan protein produced by the population of chondrocytes in the cellular composite. During development of the cellular composite described herein, the present inventors found that whilst expression of ACAN by chondrocytes is often observed (as seen for example by qPCR), this does not translate to the presence of ACAN protein as such, as shown in more detail in the Examples section of the present disclosure. ACAN is the major proteoglycan in the articular cartilage. This protein is important in the proper functioning of articular cartilage because it provides a hydrated gel structure (via its interaction with hyaluronan and link protein) that endows the cartilage with load-bearing properties. It is also crucial in chondroskeletal morphogenesis during development. Accordingly, it will be appreciated that without the presence of ACAN protein, a cellular composite merely comprising chondrocytes and osteoblasts may not be a suitable model for articular cartridge.

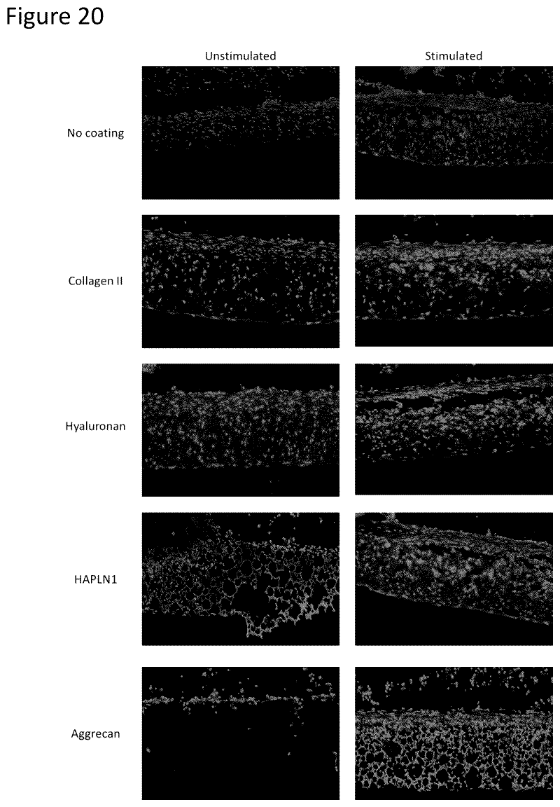

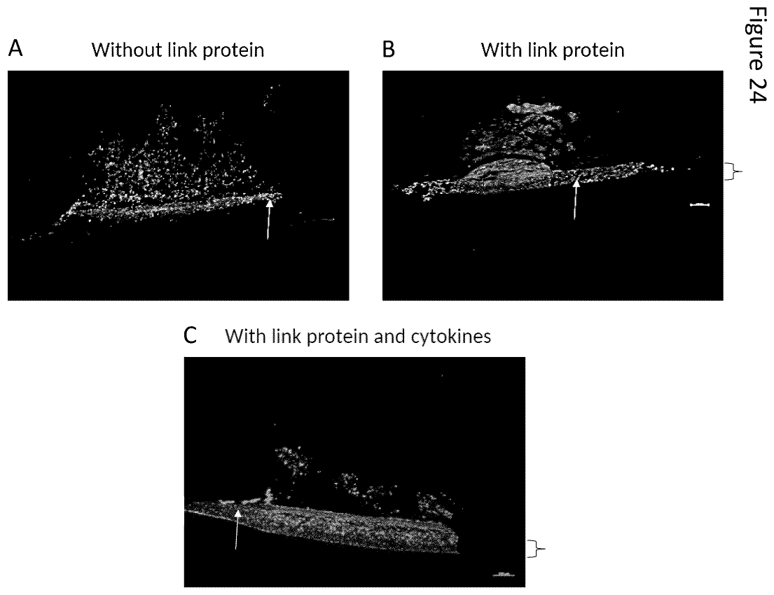

The inventors developed a novel method of making a cellular composite that comprises ACAN produced by the chondrocytes of the composite. This method involves culturing chondrocytes in the presence of a protein of the hyaluronan and proteoglycan binding link protein (HAPLN) family, such as HAPLN1. As discussed in the Examples section below and shown in FIG. 20 and FIG. 24, differentiating chondrocytes in the presence of HAPLN1 resulted in significant amounts of ACAN protein being produced.

The inventors also found that aggrecan protein may be added to the cellular composite by coating the 3D cell growth material prior to distributing a population of mesenchymal stem cells (MSCs) within a 3D cell growth material with aggrecan. However, the inventors have surprisingly found that cytokine stimulation of such cellular constructs does not result in consistent degradation of aggrecan which is a hallmark of articular cartilage disease. Therefore, whilst such aggrecan coated cellular composites may be useful as healthy models of articular cartilage, the inventors believe that cellular composites obtained by a method including the step of culturing chondrocytes in the presence of HAPLN1 in order to differentiate them may be result in better model to study diseased articular cartilage.

Furthermore, the inventors of the cellular composite described herein found that, the cellular composite, when not exposed to cytokines, does not show signs of pathogenic mineralisation. Specifically, through staining for osteocalcin the inventors have demonstrated that mineralisation markers are present only in the osteoblast region of the cellular composite and that there is a clear osteochondral interface, which is similar to what is observed in a healthy articular junction. In vivo advancement of calcification is attributed to age or disease. However, many cellular composites known in the art also observe a calcified zone at the osteochondral interface. The present inventors believe that such cellular composites are unlikely to be good models of healthy articular cartilage, as they don't replicate what happens in vivo. The present inventors believe that the lack of pathogenic mineralisation is a result of the time point at which the two cell types of the composite are co-cultured. Without wishing to be bound by this hypothesis, the inventors believe that by co-culturing the two cell types derived from stem cells after a prolonged differentiation period prior to co-culture enables the two cell types to differentiate into cell types that may more accurately resemble the cells of in vivo articular cartilage. The cellular composites described herein, which may be obtained by the methods described herein, are useful as in vitro models for studying the physiology or pathophysiology (such as osteoarthritis and/or osteoarthrosis) of articular cartilage, as well as in vitro models of healthy or diseased articular cartilage. Accordingly, they may also be useful in the context of identifying agents for treatment or prevention of articular cartilage disease as described in more detail herein.

### A Cellular Composite

Accordingly, in one aspect the present invention provides a cellular composite comprising a 3D (three dimensional) cell growth material within which a population of chondrocytes is distributed, and which has a surface that is coated with a population of osteoblasts.

The term “cellular composite” as used herein refers to an isolated artificial cell structure, i.e. not naturally occurring in the human or animal body. The cellular composite provides an ex vivo organ structure that closely represents in vivo organ structure (for example articular cartilage). The cellular composite may be a model of healthy or diseased articular cartilage, depending on the expression levels of markers such as aggrecan, SOX9, ADAMTS5, MMP9 and MMP13. In the context of the present disclosure, the cellular composite may be referred to as the “model”, “two-layer model” or “OA model”. It will be appreciated that when reference is made to an “OA model” this refers to a cellular composite that has been treated with cytokines IL1-α and/or oncostatin M. By the same token, when reference is made to the “model” or “two-layer model”, this refers to a cellular composite that has not been treated with cytokines IL1-α and/or oncostatin M.

The cellular composite comprises a 3D cell growth material (also referred to herein as the cell growth material) within which a population of chondrocytes is distributed, and which has a surface that is coated with a population of osteoblasts. Suitably, within the population of osteoblasts there may be present some chondrocyte cells. Thus, it can be said that the 3D cell growth material has a surface that is coated with a population of osteoblasts and a population of chondrocytes. The number of chondrocytes within the layer of cells coating the surface of the 3D cell growth material may be lower than the number of osteoblasts. Merely by way of example, the number of chondrocytes within the layer of cells coating the surface of the 3D cell growth material may account for about 1%, about 2%, about 3%, about 4%, about 5%, about 6%, about 7%, about 8%, about 9%, about 10%, or more of the total number of cells coating the 3D cell growth material. Suitably, the number of chondrocytes within the layer of cells coating the surface of the 3D cell growth material may account for 20% or less, 15% or less, 10% or less, or 5% or less, of the total number of cells coating the 3D cell growth material.

The “3D cell growth material” (or “cell growth material”) is the component within which the population of chondrocytes is located to form one layer of a two-layer model. In the context of the present disclosure, the 3D cell growth material may thus be referred to as the “first layer”. The 3D cell growth material supports 3D culture of the population of chondrocytes. By 3D culture it is meant that the chondrocytes are able to adopt their natural 3D morphology and distribution within the culture material. The 3D cell growth material provides a 3D support within which the cells are held such that the natural 3D morphology of the cells is maintained and such that the natural 3D distribution of cells is supported. The cells can proliferate in three dimensions within the 3D cell growth.

The 3D cell growth material may be between about 10-1000 μm thick, or between about 50 to about 500 μm, or between about 100 to 300 μm. Suitably the 3D cell growth material (for example Alvetex) is 200 μm thick.

The second layer of the two-layer model is formed by a population of osteoblasts that coat a surface of the cell growth material. However, as mentioned elsewhere, the second layer may further comprise a population of chondrocytes. In such an embodiment, the chondrocytes may account for a very small proportion (for example less than 10%, less than 5%, less than 3%, or less than 1%) of cells in the second layer. Therefore, the second layer may substantially consist of a population of osteoblasts.

Suitably, the 3D cell growth material may be synthetic (i.e. man-made). Suitably, the synthetic 3D cell growth material is inert (i.e. chemically inactive). Suitably the 3D cell growth material does not comprise a xenobiotic. Suitable, the 3D cell growth material does not comprise any proteins or fragments thereof. Suitably, the 3D cell growth material does not comprise any proteins or fragments of proteins naturally present in the extracellular matrix, such as collagen, fibrin, and/or fibronectin. Alternatively the 3D scaffold may be coated with aggrecan protein. In such an embodiment, the 3D scaffold may have been inert prior to being coated with aggrecan.

The 3D cell growth material may be formed from a scaffold or a gel.

In an embodiment where the 3D cell growth material is a gel, the gel may be a hydrogel. The gel may be selected from for example HydroMatrix™ Peptide Hydrogel, MaxGel™ Human ECM, Hystem® Stem Cell Culture, Geltrex®, or Matrigel™. The gel may comprise a biopolymer. Suitably, the biopolymer is selected from those that are commonly present in the in vivo extracellular matrix, or a combination of those that are commonly present in the in vivo extracellular matrix.

In an embodiment where the 3D cell growth material is a scaffold, the scaffold suitably comprises pores (i.e. is a porous scaffold). It will be appreciated that the pores shall be of a sufficient size to allow chondrocyte infiltration to the pores. Thus the pores are at least the size of a chondrocyte. Suitably, the pores may be between about 25-500 μm, or between about 100-300 μm, or between about 150-250 μm. Suitably the pores are between about 150-250 μm, between about 50-150 μm or between about 25-50 μm. The scaffold may comprise a porosity of over 50%, over 60%, over 70%, over 80% or over 90%.

The scaffold may be formed from a polymer. The polymer may be selected from those polymers that are suitable for cell culture. Suitably the polymer is inert. The polymer may be selected from, for example, polystyrene, Teflon®, polycarbonate, polyester, or acrylate. Suitably the scaffold is formed from polystyrene.

The scaffold may comprise a foamed (i.e. having a foam-like appearance) material. Suitably the scaffold is a foamed polymer. Suitably the scaffold is foamed polystyrene. More suitably, the scaffold material is the Alvetex® material.

The cell growth material is suitable to rest within a culturing vessel. Merely by way of example, the cell culture vessel is a 96-well, 24-well, 12-well, or 6-well cell culture plate.

The terms “population of chondrocytes” or “chondrocytes” as used herein refers to a plurality of chondrocytes, which are specialised cells that are naturally only found in cartilage. In vivo, these cells produce and maintain components of cartilage tissue, such as collagen and proteoglycans. The terms “population of osteoblasts” or “osteoblasts” as used herein refers to a plurality of osteoblasts, which produce osteoid. In vivo these cells form new bone. In vitro, a population of osteoblasts may be identified by the presence of the osteocalcin marker.

Suitably, the population of chondrocytes and/or osteoblasts may be derived from mesenchymal stem cells (MSCs). The MSCs may be immortalised (for example Y201 cells) or primary. Suitably the chondrocytes cells and/or osteoblast cells may be non-immortalised (i.e. primary) or immortalised. By primary it is meant cells that have a finite proliferation/division potential and can typically undergo from 2 up to 60 passage cycles. By immortalised it is meant cells that have acquired the ability to proliferate indefinitely, typically by transformation.

Suitably the cells may be human. It will be appreciated that human cells may be derived from human MSCs that have been differentiated into the population of chondrocytes and/or osteoblasts.

In the cellular composite of the present invention, the population of chondrocytes is distributed within the cell growth material meaning that the cells are located within the cell growth material. In an embodiment where the cell growth material is a gel (for example a hydrogel) the chondrocytes may be said to be located within the cell growth material when they are fully or partially encapsulated by the gel. In an embodiment where the cell growth material is a scaffold, the chondrocytes may be said to be located within the cell growth material when they are fully or partially in the pores of the scaffold (i.e. on the surface of the 3D cell growth material that defines the pores of the scaffold).

Suitably, the chondrocytes may produce aggrecan protein. Accordingly, the cellular composite may comprise aggrecan protein. The present inventors believe that the ability of chondrocytes to produce aggrecan is due to the method of producing the cellular composite that the inventors developed and which is described herein. This method may involve differentiating MSCs into chondrocytes in a cell culture medium comprising a protein of the HAPLN family (such as HAPLN1). Suitably, the chondrocytes may produce significant levels of aggrecan protein as seen in FIG. 24. Suitably, the chondrocytes may produce aggrecan to levels comparable (+/−10%) to those produced by chondrocytes in vivo.

The cell growth material has a surface that is coated with a population of osteoblasts. By “coated” in this context it is meant that the osteoblasts form a cultured cell sheet (or “film”) on a surface of the cell growth material. In the context of the present disclosure, this cultured cell sheet may be referred to as “the second layer”. The cultured cell sheet may be continuous, substantially continuous or discontinuous. In areas of the surface where there are pores, the osteoblasts may fill such pores.

Suitably, when the cell growth material is placed in a cell culture vessel (for example a 96-well plate) the surface that is coated with the osteoblasts is the surface that is directly opposite the surface that is in contact with (or closest to) the bottom surface of the culture vessel. In other words, the surface that is coated may be referred to as the top surface, top outer surface, or outer surface.

The surface may be coated by a monolayer of osteoblasts or multiple layers of osteoblasts, for example two, three, four, five, six, seven, eight, nine, ten or more layers. In other words, the surface of the 3D cell growth material may comprise a mono- or multi-layer osteoblast cell sheet. In the context of the present invention, it will be appreciated that such a cell sheet will not itself comprise a 3D cell growth scaffold as described herein. Suitably, the sheet will not comprise a synthetic 3D cell growth scaffold.

The surface may be coated by osteoblasts by providing said osteoblasts on the surface, or by providing MSCs and differentiating them into osteoblasts whilst on the surface. Preferably, by providing osteoblasts on the surface. In some embodiments, the surface may comprise chondrocyte cells. Therefore, the osteoblasts may be provided directly and/or indirectly on the surface. The osteoblasts may be said to be provided directly on the surface, when the osteoblasts are brought into contact with the surface. The osteoblasts may be said to be provided indirectly on the surface, when the osteoblasts are provided on chondrocyte cells that are directly on the surface of the 3D cell growth material. Suitably, when the osteoblasts are provided indirectly on the surface (due to the presence of the chondrocytes on the surface) it can be said that the surface is coated with a population of osteoblasts and a population of chondrocytes.

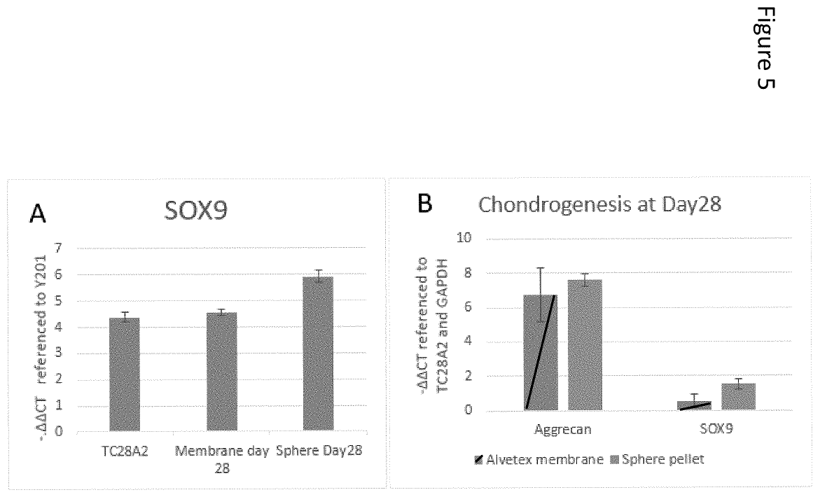

Suitably, the population of chondrocytes may have normal expression levels of a protein selected from the group consisting of SOX9, ACAN, ADAMTS5, MMP9 and/or MMP13. By “normal expression levels” it is meant expression levels that are substantially the same as expression levels in healthy chondrocytes. It will be appreciated that healthy chondrocytes may not express ADAMTS5, MMP9 and/or MMP13. Therefore, in this context, the term “normal expression level” may mean no expression or low expression of a protein selected from the group consisting of ADAMTS5, MMP9 and MMP13. Suitably, healthy chondrocytes may be chondrocytes that have not been exposed to osteoarthritic inducing cytokines selected from the group consisting of IL1-α and oncostatin M, and/or have been obtained from a healthy subject (in this context a healthy subject may be a subject that does not have osteoarthritis). Suitably, the population of chondrocytes may have substantially the same expression levels of SOX9 as TC28A2 cells, and/or increased expression of ACAN as compared to TC28A2 cells, as shown in FIG. 5B. It will be appreciated that in such an embodiment, the cell composite of the present invention may be used an in vitro model of articular cartilage, in particular an in vitro model of healthy articular cartilage. Accordingly, the present invention also provides use a cellular composite of the invention as an in vitro model of articular cartilage. Suitably, the articular cartilage may be healthy. The present invention also provides an in vitro model for studying the physiology of articular cartilage comprising the cellular composite of the invention.

Suitably, the population of chondrocytes may have abnormal expression levels of a protein selected from the group consisting of ACAN, SOX9, ADAMTS5, MMP9 and MMP13. By “abnormal expression levels” it is meant expression levels that are different to expression levels in healthy chondrocytes. The different expression levels may be higher or lower than the expression levels of healthy chondrocytes. For example, in the context of ACAN and SOX9 the expression levels may be decreased as compared to healthy chondrocytes. Merely by way of example, a decreased level may be at least 1-fold, at least 2-fold, at least 3-fold, at least 4-fold, at least 5-fold, at least 6-fold, at least 7-fold, at least 8-fold, at least 9-fold, at least 10-fold less than the expression level of healthy chondrocytes. In the context of ADAMTS5, MMP9 and/or MMP13, the expression levels may be increased as compared to healthy chondrocytes. Merely by way of example, an increased level may be at least 0.5-fold, at least 1-fold, at least 2-fold, at least 3-fold, at least 4-fold, at least 5-fold, at least 6-fold, at least 7-fold, at least 8-fold, at least 9-fold, at least 10-fold more than the expression level of healthy chondrocytes. Such abnormal expression levels may be as a result of the cellular composite of the invention being exposed to cytokines selected from the group consisting of IL1-α and oncostatin M. It will be appreciated that in such an embodiment, the cell composite of the present invention may be used an in vitro model of articular cartilage, in particular an in vitro model of diseased (for example osteoarthritic) articular cartilage. Accordingly, the present invention also provides use a cellular composite of the invention as an in vitro model of articular cartilage. Suitably, the articular cartilage may be diseased. The present invention also provides an in vitro model for studying the pathophysiology of articular cartilage comprising the cellular composite of the invention. A cellular composite that has abnormal expression levels of a protein selected from the group consisting of ACAN, SOX9, ADAMTS5, MMP9 and MMP13 may be referred to therein as the “OA model”.

ACAN is the gene that encodes the protein aggrecan which is a proteoglycan expressed in variant forms through alternative splicing. With type II collagen, it is a major component of articular cartilage and other cartilaginous tissues.

SOX9 is the gene that encodes the protein transcription factor SOX-9, which recognises the sequence CCTTGAG along with other members of the HMG-box class DNA-binding proteins. It is expressed by proliferating but not hypertrophic chondrocytes that is essential for differentiation of precursor cells into chondrocytes and, with steroidogenic factor 1, regulates transcription of the anti-Mullerian hormone (AMH) gene.

ADAMTS5 is the gene that encodes the protein disintegrin and metalloproteinase with thrombospondin motifs 5, which is a metalloproteinase that plays an important role in connective tissue organization, development, inflammation, arthritis, and cell migration.

ADAMTS5 is an extracellular matrix (ECM) degrading enzyme that show proteolytic activity toward the hyalectan group of chondroitin sulfate proteoglycans (CSPGs) including aggrecan, versican, brevican and neurocan.

MMP9 is the gene that encodes the protein matrix metallopeptidase 9 (or gelatinase-B), and functions in the degradation of type IV and V collagens.

MMP13 is that encodes the protein collagenase 3, which is an enzyme. It is involved in the breakdown of extracellular matrix in normal physiological processes, such as embryonic development, reproduction, and tissue remodelling, as well as in disease processes, such as arthritis and metastasis. Most MMPs are secreted as inactive proproteins which are activated when cleaved by extracellular proteinases. This protein cleaves type II collagen more efficiently than types I and Ill. It may be involved in articular cartilage turnover and cartilage pathophysiology associated with osteoarthritis.

Suitably, the composite may comprise a further population of cells. The further population of cells may be distributed within the cell growth material or on a surface of the cell growth material. Suitably, the further cell population may be coated directly on the cell growth material (for example mixed within the population of osteoblasts, and/or coated on the opposite surface to the surface coated with osteoblasts) or indirectly (i.e. substantially not touching the surface of the cell growth material for example by being separated from by the cell growth material by a population of osteoblasts coating the surface. Merely by way of example, the further population of cells may comprise or consist of neurons and/or Schwann cells. Suitably, neurons and/or Schwann cells may be iPSC derived cells. A cell composite comprising a population of cells such as neurons and/or Schwann cells may be used as a pain model (for example model of pain in osteoarthritis).

### Method of Producing a Cell Composite of the Invention

In one aspect, the invention provides a method of producing a cell composite comprising:
a) distributing a population of chondrocytes within a 3D cell growth material; and
b) coating a surface of the 3D cell growth material with a population of osteoblasts.

The population of chondrocytes may be distributed directly or indirectly. By directly it is meant that chondrocytes (mature chondrocytes) are deposited on or in the 3D cell growth material. By indirectly it is meant that MSCs are deposited on or in the 3D cell growth material and subsequently differentiated into chondrocytes. Thus the step of distributing a population of chondrocytes may comprise depositing a population of mesenchymal stem cells (MSCs) within the 3D cell growth material and differentiating the MSCs into chondrocytes.

Suitably the population of chondrocytes has been or may be differentiated from MSCs in a cell culture medium supplemented with a protein of the hyaluronan and proteoglycan binding link protein (HAPLN) family. Suitably the protein of the HAPLN family may be selected from the group consisting of HAPLN1, HAPLN2, HALPN3 and HAPLN4. Suitably the protein of the HAPLN family is HAPLN1. “HAPLN1” as used herein refers to hyaluronan and proteoglycan link protein 1, which is a protein that in vivo stabilizes the aggregates of proteoglycan monomers with hyaluronic acid in the extracellular cartilage matrix. Suitably HAPLN1 may have (i.e. may comprise or consist of) a sequence that is at least 80% identical to SEQ ID NO: 1. For example, has at least 85%, at least 90%, at least 91%, at least 92%, at least 93%, at least 94%, at least 95%, at least 96%, at least 97%, at least 98%, at least 99%, or 100% identical to SEQ ID NO: 1. Herein, HAPLN1 may be referred to as “link protein”.

Suitably, the cell culture medium may be supplemented with the protein of the HAPLN family (such as HAPLN1) from about day 3 of the differentiation step. In the context of the present disclosure, the differentiation step begins on the day in which the MSCs are brought into contact with a chondrogenic differentiation factor (for example TGF-β3). The day in which the MSCs are brought into contact with a chondrogenic differentiation factor may be referred to as “day 0”. The term “cell culture medium” refers to an aqueous solution of nutrients which can be used for growing cells over a prolonged period of time.

Suitably, in the methods of the invention, the cell culture medium may be supplemented with the protein of the HAPLN family (such as HAPLN1) from about day 0 of the differentiation step.

Suitably, in the methods of the invention, the cell culture medium may be supplemented with the protein of the HAPLN family (such as HAPLN1) from about day 0 to about day 28 of the differentiation step.

Suitably, the methods of the invention may further comprise contacting the population of chondrocytes with IL1-α and/or oncostatin M after step b).

Suitably, the population of chondrocytes may be contacted with IL1-α and/or oncostatin M for about 1 day, 2 days, about 3 days, about 4 days, about 5 days, about 6 days, about 7 days, about 8 days, about 9 days, or about 10 days.

Suitably, the cell culture medium may be supplemented with a protein of the HAPLN family (such as HAPLN1) at a concentration of about 25 ng/ml to about 250 ng/ml, or about 50 ng/ml to about 150 ng/ml. For example the cell culture medium may be supplemented with a protein of the HAPLN family (such as HAPLN1) at a concentration of about 100 ng/ml. Suitably the concentration of a HAPLN family (such as HAPLN1) may depend upon the number of cells. Merely by way of example, the cell culture medium may be supplemented with 100ng/25×10⁶ MSCs.

Suitably, the cell culture medium supplemented with a protein of the HAPLN family (such as HAPLN1) may comprise or consist of the components listed in Table 1, optionally at the concentrations listed in Table 1.

As described elsewhere herein, the present inventors have surprisingly found that the cell composite more closely resembles an in vivo articular cartilage, and thus may be a better in vitro articular cartilage model, when the step of distributing a population of chondrocytes comprises distributing a population of mesenchymal stem cells (MSCs) within the 3D cell growth material and differentiating the MSCs into chondrocytes. Furthermore, the inventors have found that by differentiating the cells fully into chondrocytes and osteoblasts prior to co-culturing them, pathogenic mineralisation is reduced or completely prevented. Additionally, the present inventors have surprisingly found that supplementing cell culture medium in which MSCs are differentiated into chondrogenic cells with a protein of the HAPLN family, such as HAPLN1, may be advantageous for aggrecan protein production by chondrocytes and/or stability of aggrecan within the 3D cell growth material. This enabled aggrecan protein to be present within the cellular composite of the invention.

In a related aspect, the invention provides a method of producing a cellular composite comprising:
a) distributing a population of mesenchymal stem cells (MSCs) within a 3D cell growth material and differentiating the MSCs into chondrocytes in a cell culture medium supplemented with a protein of the hyaluronan and proteoglycan binding link protein (HAPLN) family; and
b) coating a surface of the 3D cell growth material with a population of osteoblasts.

Suitably, the surface of the 3D cell growth material may be coated with a population of osteoblasts after MSCs have been differentiated into chondrocytes.

In a related aspect, the invention provides a method of producing a cellular composite comprising:
a) distributing a population of chondrocytes differentiated in a cell culture medium supplemented with a protein of the hyaluronan and proteoglycan binding link protein (HAPLN) family within a 3D cell growth material; and
b) coating a surface of the 3D cell growth material with a population of osteoblasts.

The cells may be deposited on the 3D cell growth material by seeding the cells on the cell growth material or in the cell growth material with the cells. It will be appreciated that seeding the cells on the cell growth material may be particularly relevant in the context of the cell growth material being a scaffold (for example Alvetex). Depositing the cells in the cell growth material (for example by mixing the cells with the 3D cell growth material) may be particularly relevant in the context of the cell growth material being a gel (for example hydrogel).

The chondrocytes may be distributed within the 3D cell growth material by standard cell culture techniques, or by bioprinting the cells onto or with the 3D cell growth material. When the cell growth material is a gel, the gel may be bioprinted with the chondrocytes (as the bioprinted gel may encapsulate the chondrocytes).

It will be appreciated by a person skilled in the art that in the context of a scaffold, the phrase “seeding the cells in the cell growth material” may refer to placing the cells on the surface of the cell growth material and allowing the cells to penetrate the pores of the scaffold. Penetration may be aided by gravity which will pull the chondrocytes (or MSCs) into the scaffold, allowing the cells to be dispersed through the thickness of the 3D cell growth material. However, it will be also appreciated that some chondrocytes and/or MSCs may remain on the surface of the 3D cell growth material. These cells may then differentiate and/or proliferate on the surface of the 3D cell growth material. As a result, the population of osteoblasts that coats the 3D cell growth material may comprise chondrocytes.

The cells may be deposited in numbers lower than that of the cell population distributed within the final cellular composite as defined hereinabove to allow for cell proliferation and growth.

The cell population deposited on the 3D cell growth material may comprise a total cell count of between about 15,000 to about 3,000,000 cells/cm² of the surface area of the 3D cell growth material, or between about 100,000 to about 2,500,000 cells/cm², or between about 500,000 to about 2,000,000 cells/cm², or between about 750,000 to about 1,750,000 cells/cm², or between about 1,000,000 to about 1,500,000 cells/cm² of the 3D cell growth material (for example Alvetex).

In an embodiment where MSCs are deposited in the 3D cell growth material, the cells may be cultured for a first time period in the presence of a first medium, and optionally cultured for a second time period in the presence of a second medium.

In an embodiment, where the MSCs are cultured in the presence of only the first medium, the first medium may be chondrogenic medium. In such an embodiment, the first time period may be from about 10 days to about 30 days, from about 15 days to about 25 days, or for about 21 days.

In an embodiment, where the MSCs are cultured in the presence of the first medium and second medium, the first medium may be DMEM and the second medium may be chondrogenic medium. In such an embodiment, the first time period may be from about 1 hour to about 48 hours, from about 6 hours to about 36 hours, or about 24 hours. The second time period may be from about 10 days to about 30 days, from about 15 days to about 30 days, or for about 21 to 28 days.

The term chondrogenic medium as used herein refers to a medium that supports the differentiation of MSCs to chondrocytes. Suitably, the chondrogenic medium may be serum-free DMEM. Suitably the serum-free DMEM may comprise ascorbic acid phosphate at a final concentration of about 50pg/ml, dexamethasone at about 10 nM, L-proline at final concentration of about 40pg/ml, about 1% ITS+, with TGF-β3 at about 10 ng/ml.

Upon distributing the population of chondrocytes within a 3D cell growth material, the surface of the 3D cell growth material may be coated with a population of osteoblasts. Suitably, the coating of the surface may include depositing from about 100,000 to about 1,000,000, suitably about 500,000 cells/cm² of the surface.

Upon depositing the population of osteoblasts on the surface, the osteoblasts may be incubated for about 1 to 10 hours, or about 2 to 7 hours, suitably about 3 hours at 37° C. to allow the cells to adhere to the surface. The cellular composite with the deposited chondrocytes and attached osteoblasts may be then immersed in cell culture media. Suitably, it may be immersed in a mixture of two different cell culture media. For example, the first medium may be DMEM with 10% fetal bovine serum (FBS). The second medium may be, for example, osteochondral medium. Osteochondral medium (OC) may comprise DMEM with 2% FBS, L-Ascorbic acid-2-phosphate (final concentration at 50pg/I), dexamethasone at 10 nM, L-proline at final concentration of 40pg/ml and 1% insulin, transferrin and selenium additives.

The osteoblasts for depositing on the surface may be differentiated from MSCs (suitably human MSCs). Methods for differentiating MSCs into osteoblasts are well known in the art. Merely by way of example, MSCs (such as Y201 cells) may be seeded in a cell culture flask (for example 75 ml flask) in DMEM until about 80% confluent. Once this confluency is reached, osteogenic media may be added for about 21 to about 28 days. The osteogenic media may be a high glucose DMEM comprising L-Ascorbic acid-2-phosphate (final concentration at 50pg/I), B-Glycerophosphate at 5 mM and dexamethasone at 10 nM. The media may be replenished twice weekly.

Suitably, the chondrocytes may be fully differentiated prior to step b) of methods described herein being carried out. Fully differentiated chondrocytes may be identified by the skilled person using a number of known methods, for example by determining gene expression of ACAN and/or SOX9, and/or aromatase immunohistochemistry. Suitably, chondrocytes may be fully differentiated upon being cultured in chondrogenic medium for at least 7 days, at least 14, at least 21 day, or more. A “chondrogenic medium” refers to a culture medium that promotes the growth and differentiation chondrogenic cells from MSCs. Such a medium will typically comprise differentiation factors. Suitably, step b) of the methods described herein may be carried out about 7 days, about 8 days, about 9 days, about 10 days, or more after differentiation of MSCs of chondrocytes has been initiated (i.e. day 0 of the differentiation step). For example step b) may be carried about 12 days, about 14 days, about 16 days, about 18 days or more after differentiation of MSCs of chondrocytes has been initiated. For example, step b) may be carried out about 21 days or 28 days after differentiation of MSCs of chondrocytes has been initiated. Suitably, step b) of the methods described herein may be carried out after 6 days, after 7 days, after 8 days, after 9 days, after 10 days, after 11 days, after 12 days, after 13 days, after 14 days, after 15 days, after 16 days, after 17 days, after 18 days, after 19 days, after 20 days, after 21 days, after 22 days, after 23 days, after 24 days, after 25 days, after 26 days, after 27 days, after 28 days, or more after differentiation of MSCs of chondrocytes has been initiated.

Suitably, the when step b) of the methods described herein is performed, the protein from the HAPLN family (for example HAPLN1) may no longer be present in the cell culture medium in which the chondrocytes are cultured. Accordingly, prior to step b) the method may comprise exchanging the medium supplemented with the protein from the HAPLN family (for example HAPLN1) with a medium not supplemented with the protein from the HAPLN family (for example HAPLN1).

As mentioned elsewhere in the present description, the chondrocytes may have normal or abnormal expression of a protein selected from the group consisting of ACAN, SOX9, ADAMTS5, MMP9 and MMP13. A cellular composite of the invention that has abnormal expression of a protein selected from the group consisting of ACAN, SOX9, ADAMTS5, MMP9 and MMP13 may be used as an in vitro model of diseased articular cartilage. Suitably the disease may be osteoarthritis and/or osteoarthrosis.

Suitably, the chondrocytes may have abnormal expression due to being exposed to osteoarthritic inducing cytokines. Suitably, the osteoarthritic inducing cytokines may be selected from the group consisting of IL1-α and oncostatin M.

Accordingly, in one embodiment, the method of producing a cell composite of the invention may further comprise the step of:
c) contacting the cellular composite with osteoarthritic inducing cytokines, such as those selected from the group consisting of IL1-α and/or oncostatin M.

Suitably, the step of contacting the composite with osteoarthritic inducing cytokines may involve contacting the population of chondrocytes (and optionally osteoblasts) with said osteoarthritic inducing cytokines.

Suitably, the cellular composite may be contacted with IL1-α and/or oncostatin M for between about 1-10 days, for between about 2-9 days, for between about 3-8 days, for between about 4-7 days, or for between about 5-6 days. Suitably the cellular composite may be contacted with IL1-α and/or oncostatin M for about 1 day, about 2, about 3, about 4, about 5, about 6, about 7, about 8, about 9, about 10, or more days. More suitably the cellular composite may be contacted with IL1-α and/or oncostatin M for about 1 day (i.e. about 24 hours).

Suitably, the cellular composite may be contacted with IL1-α at a concentration between about 10 ng/ml-200 ng/ml, or between about 50 ng/ml-150 ng/ml, or between about 50 ng/ml-100 ng/ml. For example, at a concentration of about 50 ng/ml, about 60 ng/ml, about 70 ng/ml, about 80 ng/ml, about 90 ng/ml, or about 100 ng/ml. More suitably, the cellular composite may be contacted with IL1-α at a concentration of about 50 ng/ml or about 100 ng/ml.

Suitably, the cellular composite may be contacted with oncostatin M at a concentration between about 1 ng/ml-50 ng/ml, or between about 5 ng/ml-20 ng/ml, or between about 10 ng/ml-20 ng/ml. For example, at a concentration of 10 ng/ml, 11 ng/ml, 12 ng/ml, 13 ng/ml, 13 ng/ml, 14 ng/ml, 15 ng/ml, 16 ng/ml, 17 ng/ml, 18 ng/ml, 19 ng/ml, or 20 ng/ml. More suitably, the cellular composite may be contacted with oncostatin M at a concentration of about 10 ng/ml or about 20 ng/ml.

Oncostatin M (also known as OSM) is a protein that in humans is encoded by the OSM gene OSM is a pleiotropic cytokine that belongs to the interleukin 6 group of cytokines.

Interleukin 1 alpha (IL-1a) also known as haematopoietin 1 is a cytokine of the interleukin 1 family that in humans is encoded by the IL1A gene.

Suitably, when the cellular composite may be contacted with IL1-α and/or oncostatin M, the cell composite may be in OC (osteochondral) media or DMEM media.

In a further aspect, the invention provides a cell composite produced by a method of the invention.

The method of producing a cellular composite as described herein may comprise distributing a population of neurons and/or Schwann cells. Suitably, the population of neurons and/or Schwann cells may be distributed within the 3D cell growth material simultaneously with distributing a population of mesenchymal stem cells (MSCs) within a 3D cell growth material. Alternatively, or additionally, the population of neurons and/or Schwann cells may be distributed within the 3D cell growth material before and/or after distributing a population of mesenchymal stem cells (MSCs) within a 3D cell growth material.

In a further aspect, the present invention provides a method of producing a cellular composite comprising:
a) distributing a population of mesenchymal stem cells (MSCs) within a 3D cell growth material and differentiating the MSCs into chondrocytes in a cell culture medium; and
b) coating a surface of the 3D cell growth material with a population of osteoblasts.

Suitability, the 3D cell growth material may be coated with aggrecan prior to step a). Optionally, the method may comprise the step differentiating the MSCs into chondrocytes in a cell culture medium comprising a protein of the HAPLN family (such as HAPLN1).

In a further aspect, the present invention provides a cellular composite obtained by a method comprising the step of:
a) distributing a population of mesenchymal stem cells (MSCs) within a 3D cell growth material and differentiating the MSCs into chondrocytes in a cell culture medium; and
b) coating a surface of the 3D cell growth material with a population of osteoblasts, wherein the 3D cell growth material may be coated with aggrecan prior to step a). Suitably such a cellular composite used as an in vitro model for studying the physiology or pathophysiology of articular cartilage.

### Method of Screening

In one aspect, the invention provides a method of screening an agent for the treatment or prevention of articular cartilage disease, comprising:
a) providing a cellular composite of the invention;
b) exposing the cellular composite to the agent; and
c) determining whether the agent has a therapeutic effect on the composite.

The term “articular cartilage disease” as used herein refers to a disease which may be associated with and/or result in articular cartilage damage. Suitably an articular cartilage disease may be selected from the group consisting of osteoarthritis, osteoarthrosis, and rheumatoid arthritis.

In the context of the present invention, an agent may be said to have a therapeutic effect if it has the ability to treat or prevent articular cartilage damage (for example osteoarthritic changes) in the cellular composite of the invention. Such articular cartilage damage or osteoarthritic changes (also referred to herein as changes associated with osteoarthritis) may include decreased expression of ACAN and/or SOX9, and/or increased expression of ADAMTS5, MMP9 and/or MMP13 in chondrocytes of the composite upon exposure of the composite to IL1-α and/or oncostatin M, and/or reversal or prevention of the separation of the two layers of the OA model.

It will be appreciated that in the context of a method of screening an agent for the treatment of an articular cartilage disease, e.g. osteoarthritis, it may be desirable for the population of chondrocytes of the cellular composite to have abnormal expression of a protein selected from the group consisting of ACAN, SOX9, ADAMTS5, MMP9 and MMP13. As mentioned elsewhere in the present description, abnormal expression may be decreased expression of ACAN and/or SOX9, and/or increased expression of one or more of ADAMTS5, MMP9 and MMP13 as compared to a healthy chondrocyte. It will equally be appreciated that in the context of a method of screening an agent for the prevention of articular cartilage disease, e.g. osteoarthritis, it may be desirable for the population of chondrocytes of the cellular composite to have normal expression of a protein selected from the group consisting of ACAN, SOX9, ADAMTS5, MMP9 and MMP13. The term “normal expression” is defined elsewhere in the present disclosure.

The term “treatment” as used herein refers to a complete or partial reversal of changes in the cellular composite associated with articular cartilage disease, e.g. osteoarthritis, and/or prevention, or complete or partial reversal of pain associated with such changes. Merely by way of example, these changes may include increased expression of ACAN and/or SOX9, and/or decreased expression of ADAMTS5, MMP9 and/or MMP13. The term “prevention” as used herein refers to a complete or partial reduction of the development of changes associated with articular cartilage disease, e.g. osteoarthritis. As shown in the Examples section of the present disclosure, and as discussed hereinabove, exposure of the cellular composite to IL1-a and/or oncostatin M results in the chondrocytes of the composite reduced expression of ACAN and/or SOX9, and/or increased expression of one or more of ADAMTS5, MMP9 and/or MMP13. An agent that prevents the development of changes associated with articular cartilage disease, e.g. osteoarthritis, may reduce the changes in expression caused by exposure of the composite to IL1-α and/or oncostatin M.

Whether there is a complete or partial reversal of pain may be determined by measuring for example release of neurotransmitters such as Substance P from the neuronal cells that may be present in the cellular composite.

The skilled person will be aware of other assays and outputs (other than the expression of ACAN, SOX9, ADAMTS5, MMP9 and/or MMP13) that may be indicative of whether a test agent may useful for treatment or prevention of articular cartilage disease. Such other assays may include measuring MMP activity.

Suitably, the agent being screened may be administered to the cell composite in an amount sufficient to and for a time necessary to exert a desired effect upon cell composite. These amounts and times may be determined by the skilled artisan by standard procedures known in the art.

Certain embodiments of the invention will now be described by way of reference to the following examples and accompanying figures.

### EXAMPLES

### Example 1

### 1. Materials and Methods

### 1.1 Cell Culture

MSCs (Y201) were grown in complete Dulbecco's Modified Eagle Medium (DMEM, Thermo Fisher) in horizontal culture flasks and had adherent and epithelial morphology. Complete DMEM contained 10% fetal bovine serum (FBS), 1% penicillin-streptomycin and 1% amphotericin B. The cells were used to perform chondrogenesis (in a 3D scaffold) and osteogenesis in 2D culture. Differentiated cell were then used to construct a model which consisted of two layers, an upper bone layer (osteoblasts) and a lower cartilage layer (chondrocytes).

### 1.2 Osteogenic Differentiation

MSCs (Y201) were seeded in 75 ml flasks in DMEM until 80% confluent, media was removed, and osteogenic media was added for the following 21 days which included high glucose DMEM in addition to L-Ascorbic acid-2-phosphate (final concentration at 50pg/1), B-Glycerophosphate at 5 mM and dexamethasone at 10 nM (James S, et al, 2015). Media was replenished twice weekly.

### 1.3 Chondrogenic Differentiation

MSCs (Y201) were grown in complete DMEM until 80% confluent they were then trypsinised and resuspended into 20 million cells per millilitre in DMEM media; 25 μl cell suspension (equal to 0.5 million cells) were placed in the middle of an Alvetex 3-D scaffold membrane (or Biogel) and allowed to adhere at 37° C. incubator with 5% CO₂ for 3 hours. Alvetex is a commercially available scaffold suitable for 3D culture in 96-well plates. Wells were flooded with complete DMEM media for 24 hours and media was then changed to chondrogenic media for 21 days with media replenishment twice a week. Chondrogenic media contains serum-free DMEM with L-ascorbic acid phosphate at a final concentration of 50pg/ml, dexamethasone at 100 nM, L-proline at final concentration of 40 μg/mL, 1% ITS+, with 10 ng/mL of TGF-p-3; medium was changed twice weekly (James S, et al, 2015). The MSCs were also differentiated in sphere pellet for 28 days and was used as a positive control for 3D chondrogenesis.

### 1.4 Construction of Two-Layer Model of Chondrocytes and Osteoblasts

The MSCs were differentiated in a 3D Alvetex scaffold in chondrogenic media for 21-28 days. Simultaneously, a different batch of MSCs (Y201) were differentiated to osteoblasts in osteoblastic media in 2D culture for 21 days as described above. Differentiated osteoblasts were trypsinised, counted and resuspended in DMEM media. Osteoblasts were then seeded on top of chondrocytes in the Alvetex membrane (or Biogel) and left for 3 hours at 37° C. incubator to adhere to the chondrocytes before being immersed with media for 10 days. Two kinds of media were tested for maintaining the two-layer membrane. One was complete DMEM with 10% fetal bovine serum (FBS) whereas the second media was osteochondral media which contained DMEM with 2% FBS, L-Ascorbic acid-2-phosphate (final concentration at 50 μg/l), dexamethasone at 10 nM, L-proline at final concentration of 40 μg/ml and 1% ITS+. Media was changed every two days and supernatant was frozen at −80° C.

### 1.5 Validation of the Two-Layer Model Using RNA Expression Analysis

1.5.1 RNA Isolation and cDNA Synthesis

RNA was isolated from the model using Qiagen RNAeasy mini kit (Qiagen, UK) according to the manufacturer instructions. RNA quantity was measured using the NanoDrop ND-1000 spectrophotometer (NanoDrop). The quality of RNA was determined using the Agilent 2100 Bioanalyzer (Agilent Technologies). RNA was treated with DNase I enzyme (Promega) and incubated at room temperature for 15-20 minutes according to the manufacturer's instructions. The synthesis of cDNA was established using random hexamers primers and synthesized using a high capacity cDNA kit (Applied Biosystems) with a reverse transcriptase enzyme of Moloney murine leukaemia virus (MMLV).

### 1.5.2 Real Time PCR

Real time PCR was performed using primer probes from TaqMan gene expression assay (Applied Biosystem, UK). Fast master mix was used (Applied Biosystem) and GAPDH was used as a housekeeping gene for referencing. Reactions were performed in 20 μl volumes in 96 well plates using Quant Studio 3 (Applied Biosystems) with the following cycling parameters: a hold stage with 2 minutes at 50° C. and 20 seconds 95° C., a PCR stage of 40 cycles comprising a denaturing step at 95° C. for 1 second and extension step at 60° C. for 20 seconds. Analysis was performed using ΔΔCT method and results were expressed as fold change.

### 1.6 Validation of the Two-Layer Model Using Immunofluorescence Staining

Formalin fixed paraffin embedded slides (FFPE) of the sectioned 3D model were stained for various markers using the following antibodies, rabbit anti human SOX-9 (Millipore, ABE2868), Aggrecan (Abcam, Ab3778) and Collagen2a1 (Mouse mAb, Merck). FFPE slides were dewaxed for 10 minutes in xylene (Fisher Scientific), followed by 5 minutes immersing in ethanol gradient of 100%, 90% and 70%, respectively. Antigen was retrieved by immersing slides in citric saline (pH:6) in a microwave for 10 minutes and left to cool for an additional 20 minutes at room temperature. Slides were blocked with 20% pig serum for one hour followed by application of the primary antibody overnight at 4° C. After washing, slides were incubated with the secondary antibody for two hours followed by washing, dehydration and mounting with DAPI-Mount (Abcam).

### 1.7 Embedding and Sectioning

MSCs that were differentiated in 3D Alvetex scaffold (or Biogel) for 21 days were placed in formalin 10% for 24 hours. Alvetex scaffolds were immersed in a gradient of ethanol at 30%, 50%,70%, 80%, 90%, 95% and 100% for 10 minutes each. Then they were immersed in Histoclear (National Diagnostics Ltd) for 30 minutes at room temperature (RT), and in 50% histoclear-paraffin wax at 60° C. for 30 minutes. Alvetex scaffolds were then immersed in paraffin wax only for another 30 minutes at 60° C. Following this step, the scaffolds were embedded in paraffin and blocks were generated for further generating of FFPE sections.

### 1.8 Histology Staining—to Demonstrate Two-Layer Cartilage Like Model

### 1.8.1 Haematoxylin Eosin (H&E) Staining

FFPE slides were dewaxed for 10 minutes in xylene and rehydrated by immersing in gradient concentrations of ethanol at 100%,70% and 50%, respectively for 5 minutes each. Slides were then immersed in water for 2 minutes. Afterward, slides were immersed in haematoxylin for 5 minutes, washed in tap water for 1-2 minutes and incubated in Scott's water. Slides were then incubated in eosin for 3 minutes followed by washing and dehydration.

### 1.8.2 Alcian Blue—Staining Glycosaminoglycans in Cartilage

Following dewaxing and rehydration, slides were then immersed in water for 2 minutes and stained with 1% Alcian blue in 1% acetic acid overnight at 4° C. Slides were then washed in running water for 5 minutes and counterstained with nuclear red for 3 minutes. Slides were then washed, dehydrated and mounted.

### 1.8.3 Toluidine Blue—Used to Stain Proteoglycans and Glycosaminoglycans in Tissues Such as Cartilage

Staining with toluidine blue was performed following dewaxing of FFPE slides in histoclear for 10 minutes and passing through a gradient of ethanol concentrations at 100%, 70%, and 50% for 5 minutes each. Slides were then hydrated and stained with 1% toluidine blue in 70% ethanol (pH:2.5) for 2-3 minutes. Slides were washed with tap water, dehydrated and mounted.

1.8.4 Safranin O—Used to Identify Chondrocytes that have been Derived from Both Human and Rodent Mesenchymal Stem Cells (MSCs)

First, Weiger's iron haematoxylin was prepared by mixing one volume of 10% haematoxylin-ethanol with one volume of 30% ferric chloride (1% hydrochloric acid). Safranin O was prepared as 1% Safranin O solution in distilled water. Slides were dewaxed and hydrated as mentioned above. Slides were stained in Weigert's iron Haematoxylin for 5 minutes followed by rinsing in 1% acid-alcohol for 2 seconds and washed by distilled water. Slides were then stained with 1% Safranin O for 30 min, washed and dehydrated as usual.

### 1.8.5 Alizarin Red—Commonly Used Stain to Identify Calcium Containing Osteocytes in Differentiated Culture of Both Human and Rodent Mesenchymal Stem Cells (MSCs).

Following dewaxing and hydration, slides were stained with Alizarin solution for 20-30 minutes. Slides were washed with dH2O 3 times until they are clear. Slides were then investigated for calcium crystals using colour camera on inverted microscope.

### 1.9 Induction of Osteoarthritis Disease Model Using Cytokines

Following construction of the two-layer model, cells were left to rest for three days in either DMEM or OC media. Fresh media was then added which contained IL1-α and oncostatin M (OSM) at final concentration of 50 ng/ml and 10 ng/m respectively (H. E. Barksby, et al 2006). Identically, another set of wells were incubated with both cytokines in double concentration of the amounts stated above. Control cells contained models in the same media with no added cytokines and dealt with in similar. Media was changed every two days for six days whereas supernatants were collected and stored at −80° C. for later ELISA tests. Models were then harvested and used for RNA extraction or fixed in formalin 10% for FFPE sections.

### 2. Results

### 2.1 Chondrogenesis on Alvetex Membrane

### 2.1.1 Phenotypic Changes

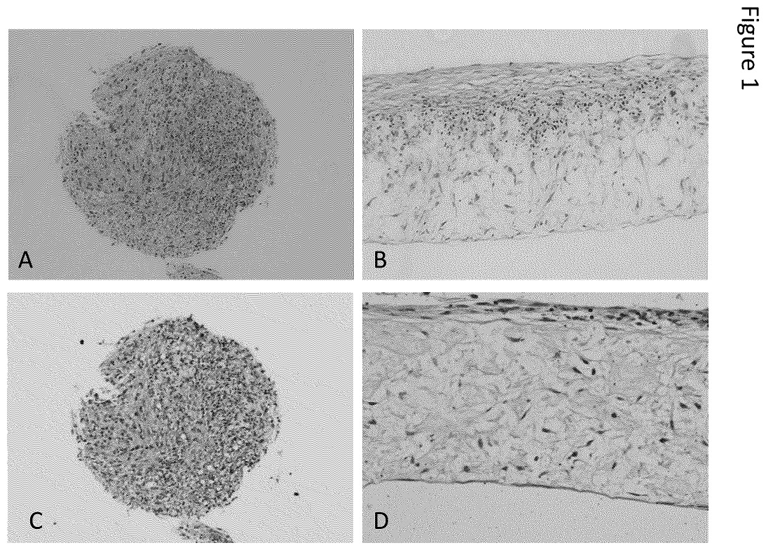

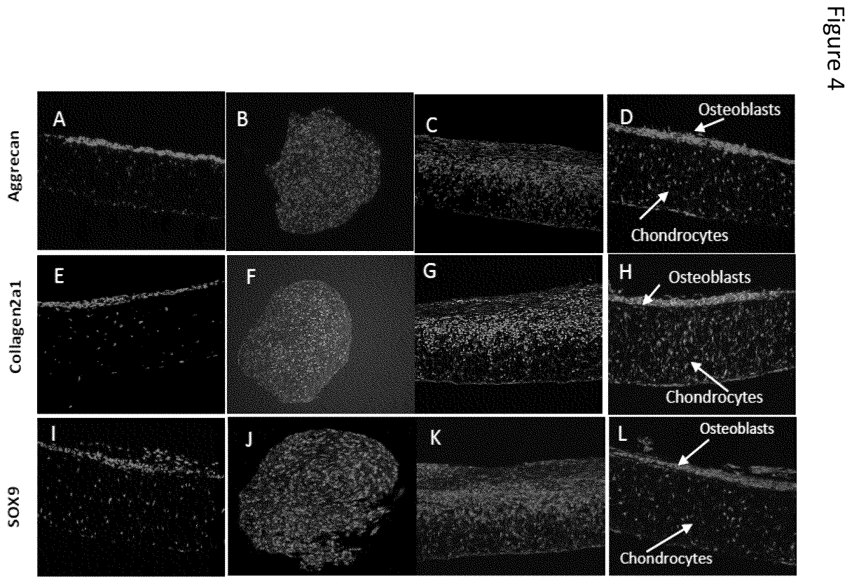

After differentiation, the MSCs were investigated using histology staining such as Alcian blue (FIG. 1D) and antibody labelling such as aggrecan, collagen2a1 and SOX9 (FIGS. 4, 5, and 6C).

### 2.1.2 Gene Expression of Generated Chondrocytes

Using quantitative real time PCR (qPCR), chondrogenic gene expression such as SOX9 was shown to be significantly increased when referenced to the housekeeping gene GAPDH. This change was further supported by increasing expression of aggrecan, the major structural proteoglycan found in the extracellular matrix of cartilage in these cells when referenced to human chondrocyte cell line TC28A2 (FIG. 5).

### 2.1.3 Model Construction

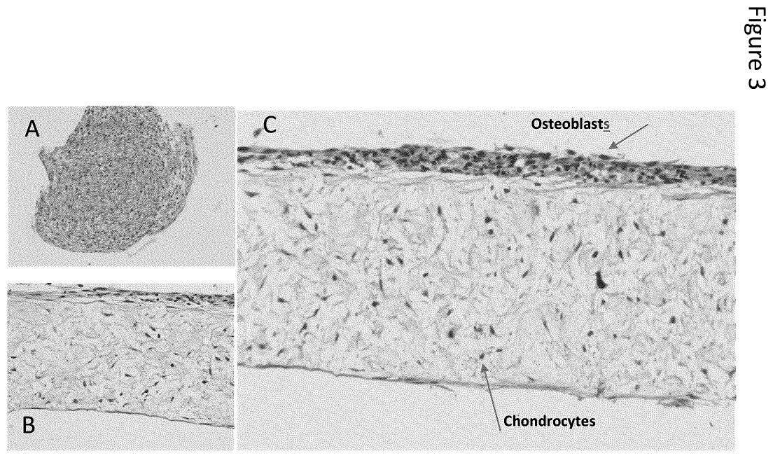

As explained above, cells were seeded on top of the Alvetex membrane and left to rest for 48 hours in DMEM media in 37° C. incubator with 5% CO2. Media was then replaced by chondrogenic media for the following 28 days where a layer of osteoblast were seeded on top of the membrane and left to rest for the following ten days in DMEM. Membrane was fixed and stained (FIG. 3).

### 2.2 Model Phenotyping and Genotyping

### 2.2.1 Alcian Blue

Two-layer model was stained with Alcian blue and compared to chondrogenesis in sphere pellet (FIG. 3A) and in 3D differentiation only (FIGS. 3B and C), i.e. without adding another layer of cells or changing the media. This figure shows chondrocytes-generated extra cellular matrices (ECM) following ten days incubation in DMEM (FIG. 3).

### 2.2.2 Antibody Staining

Two-layer models were investigated for staining with antibodies against aggrecan, collagen2a1 and SOX9. Chondrocytes differentiated inside the Alvetex membrane showed upregulation of the staining following ten days incubation in normal growth media (DMEM) alongside osteoblasts (FIGS. 4, D, H and L, respectively). Chondrocytes differentiated in sphere pellets (FIGS. 4B, F, and J) and 3D Alvetex membrane (FIGS. 4C, G, and K) were used as a positive control.

2.2.3 Chondrocytes Markers at the mRNA Level

Following 28 days differentiation in chondrogenic media, changes in genes known to be highly expressed in chondrocytes were investigated on mRNA level by quantitative real time PCR. These genes included SOX9 and aggrecan. qPCR results showed high expression in SOX9 in 3D Alvetex differentiated chondrocyte cells compared to original mesenchymal Y201 cells (FIG. 5 A). Further, when compared to human chondrocyte cell line TC28A2 and sphere pellets, these cells showed high levels of expression in SOX9 and aggrecan genes (FIG. 5 B).

### 2.3 Induction of Osteoarthritis—Generating OA Model

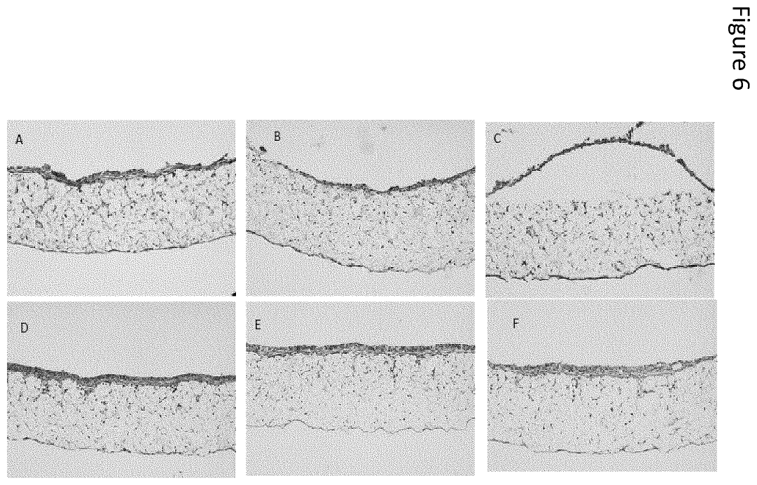

OA was induced using two concentrations cytokine namely IL1-α and oncostatin M (OSM) in DMEM/osteochondral (OC) media. Following OA induction, FFPE sections of all models were stained for Alcian blue (FIG. 6). Results show less ECM structure in OA induced models (B, C and E, F) compared to no treatment models (A, D). “OA-induced model” may also be referred to as “OA model” herein.

### 2.4 Immunofluorescence Staining for Chondrogenic Markers in OA-Induced Models

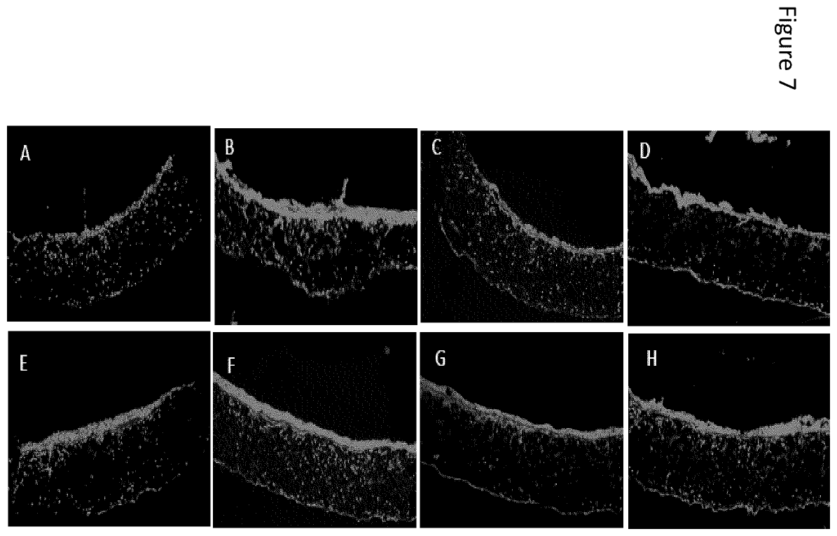

Following OA induction, aggrecan, collagen2a1 and SOX9 were investigated by immunofluorescence staining. Results show that antibody staining with aggrecan, collagen 2A1 (data not shown) and SOX9 (FIG. 7) were downregulated in OA models compared to no treatment models (i.e. models not treated with cytokines). These changes were further analysed and showed significant changes in OA models compared to no treatment models (data not shown).

### 2.5 Real Time PCR for Chondrogenic Markers

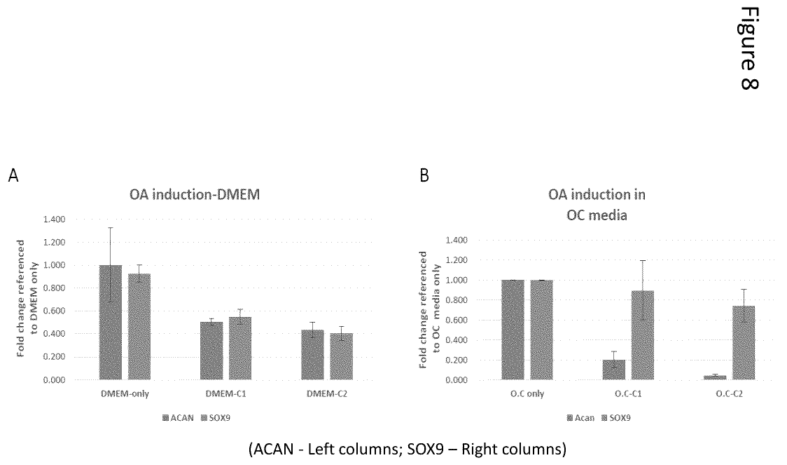

To investigate changes taking place at mRNA level following OA induction, qPCR was performed for aggrecan and SOX9 and referenced to non-treated samples which were incubated in the same media. Results show decreased expression in these genes at two concentrations and in both media cultures (FIG. 8).

Decreased expression of aggrecan and SOX 9 would infer damage had taken place in the model after induction using DMEM C1 and C2 and OC C1 and C2.

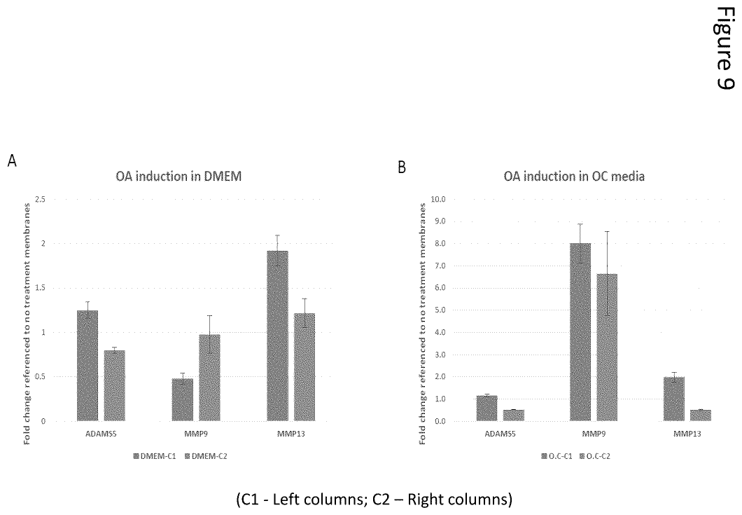

To further expand the range of gene changes, qPCR of ADAMTS5, MMP9 and MMP13 was performed. Results show that expression of these genes was increased at different ratios compared to no treatment controls (FIG. 9) following OA induction. This is indicative of damage.

The results have shown that by using cells differentiated from MSCs a two layer osteochondral in vitro model can be developed which can then be damaged by the cytokine cocktail with increased and decreased expression of the relevant markers associated with OA.

Osteochondral media and a time point of 24 hours was chosen as the best condition to induce cytokine stimulation of osteochondral model.

### 3. Use of Drugs to Reverse the OA Damage in the OA Model

A mixture of drugs including anti AdamTS4; AdamTS 5 and MMP at 10 mM and the carrier molecule DMSO (1:10000) as well as isotype IgG1 at 50 ug/ml were added 24 hours post the model construct and added at the same time as the cytokines and cultured for either 3 days or 6 days before harvesting

The OA models were then embedded and sectioned, stained with haematoxylin and eosin and by immunohistochemistry by double staining with aggrecan and osteocalcin. RTPCR were also performed on the models to observe changes in gene expression such as SOX9,MMP9 MMP13, ELISAs were also performed to observe changes in protein levels.

In Examples 2-4, the inventors detail the steps taken to further characterise and develop this model and outline the use of Primary MSCs within the OA system.

### Example 2

### 1. Characterisation of MMP Activity of OA Models Treated with a Mixture of Anti-ADAMTS-4/ADAMTS-5, and MMP Inhibitor

### 1.1 Method

Osteochondral models were constructed using methods described herein and were treated simultaneously with stimulatory cytokines (to form OA models) and treated with a small molecule consisting of a mixture of anti-ADAMTS-4/ADAMTS-5, and MMP Inhibitor (at a concentration of 1 uM). Following incubation the resulting OA models and their respective supernatants were harvested. Supernatants assessed via FRET assay to determine MMP activity. MMP activity was determined through fluorescent activity (RFU). RNA isolated from Y201 based models were also analysed via RNA sequencing for further characterization of the OA model.

### 1.2 Results

### 1.2.1 Determining the MMP Activity of OA Models

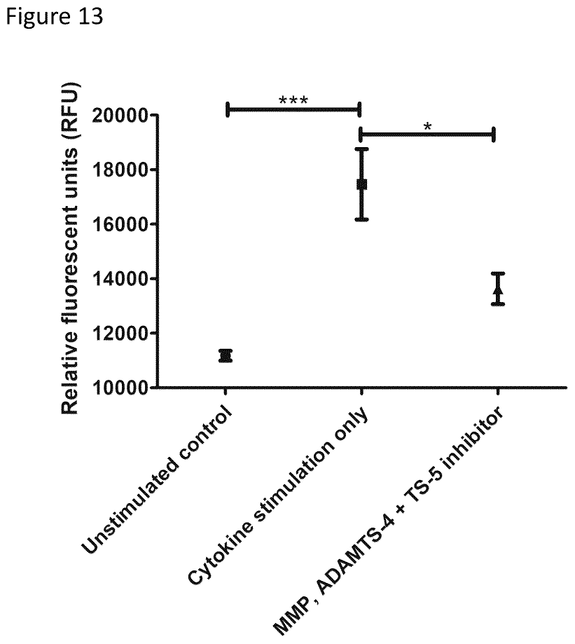

MMP activity of models versus OA models as determined via FRET assay is shown in FIG. 13. The MMP activity of unstimulated models was shown to be relatively low when compared to OA model (stimulated with cytokines) and the change in MMP was shown to be statistically significant (P<0.0001) when compared by one-way Anova. Treating OA models (cytokine stimulated) with the small molecule mixture inhibitor resulted in the reduction of MMP activity and reduction in statistical significance (P<0.05) when compared to cytokine only treated models (OA models).

### 1.2.2 Gene Expression of Models (Unstimulated) Versus OA Models (Cytokine Stimulated)

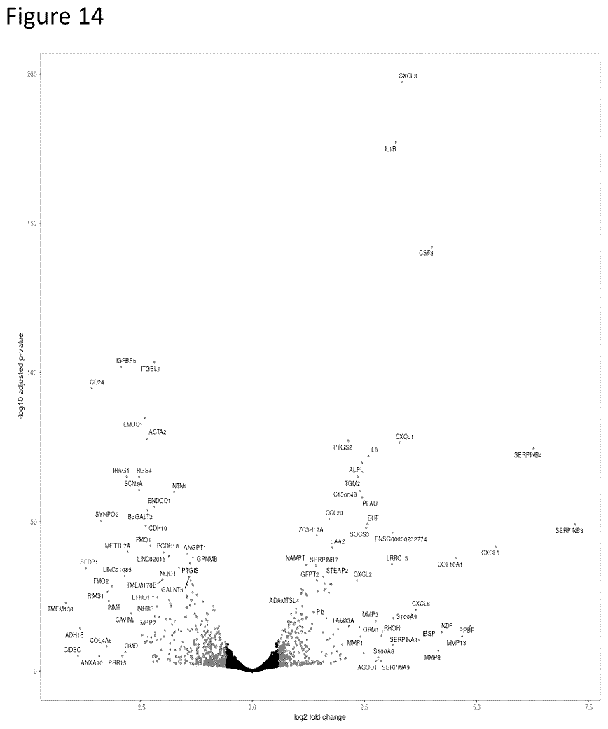

RNA sequencing was conducted, and the data presented as a volcano plot (FIG. 14). FIG. 14 shows the fold change in gene expression of OA models (cytokine stimulated) compared to models (unstimulated) with a logarithmic scale for each genes P value. The data presented in FIG. 14 clearly shows the upregulation of MMP13, MMP8, MMP3 and MMP1 in OA models. Furthermore IL-6 was upregulated in response to cytokine stimuli.

### 1.3 Summary

MMP activity is increased in response to cytokine stimulation, this activity can be altered through small molecule treatment.

### Example 3

### 1. Developing OA Models Using Primary MSCs

### 1.1 Method

Primary MSC-like bone cells were phenotypically validated through flow cytometry. A total of 3 donors were assessed and used for model development. Once the cells were phenotypically validated osteochondral models were constructed using previously established methods. Primary cell-based models were treated with stimulatory cytokines to produce OA models. Following incubation with the cytokines the resulting OA models and supernatants were harvested. RNA was extracted from harvested OA models and analysed using quantitative-PCR (q-PCR) to quantify fold change in gene expression in addition to RNA sequencing. The supernatants were analysed for MMP concentrations, MMP activity and ADAMTS4 concentration.

### 1.2 Results

### 1.2.1 the Phenotypic Validation of MSC-Like Bone Cells

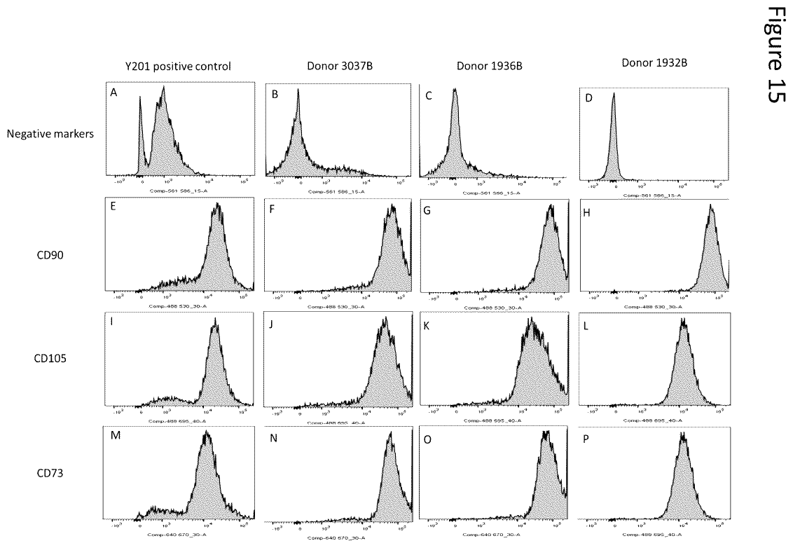

The phenotypic validation of MSC-like bone cells is presented in FIG. 15. The cells were phenotyped along Y201 MSCs which acted as a positive control. Each donor and the Y201 cells were stained for MSC markers CD90, CD105, CD73 and a cocktail of negative MSC markers. Most primary cells from all three donors did not exhibit negative MSC markers (FIG. 15B-D) whereas by comparison the Y201s did contain a population of cells which did express some negative MSC markers (FIG. 15A). All donors presented strong staining for MSC positive markers CD90 (FIG. 15E-H), CD105 (FIG. 15I-L), and CD73 (FIG. 15M-P).

1.2.2 q-PCR Analysis of OA Models Constructed Using Primary MSCs

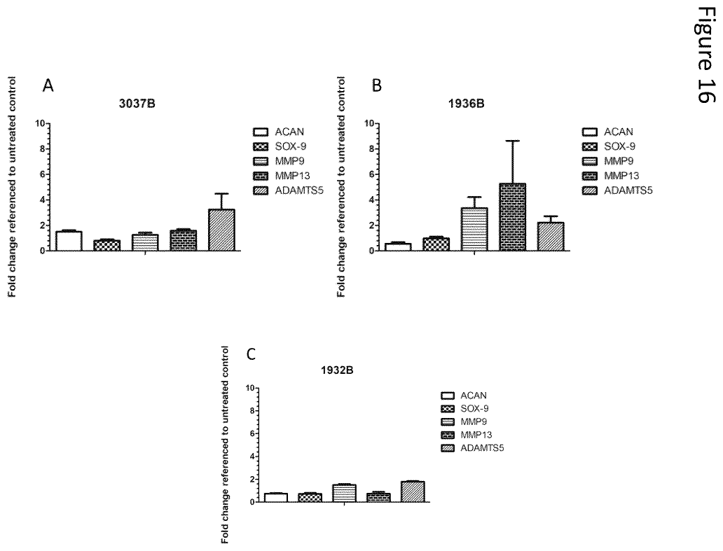

FIG. 16 shows the q-PCR analysis of OA models constructed using primary MSCs. Due to donor variability the q-PCR data was presented for each individual donor rather than as a mean for all three donors. Data is presented as fold change in gene expression of OA models (cytokine stimulated) in relation to control models (untreated). Donor 3037B (FIG. 16A) showed elevated expression of ADAMTS5 and to a lesser extent elevated MMP13 expression. Varying upregulation of all three tissue degradation related genes was observed in the gene expression analysis of donor 1936B (FIG. 16B). By comparison donor 1932B (FIG. 16C) demonstrated a lesser response at the gene expression level. FIG. 16C reveals a marginal increase in gene expression of MMP9 and ADAMTS5 following cytokine stimulation of donor 1932B.

### 1.2.3 Levels of MMP13 and ADAMTS4 in the Supernatant of Harvested OA Models

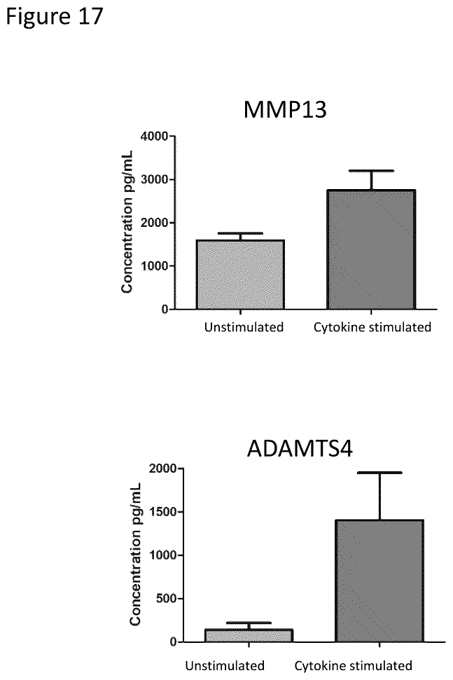

Levels of MMP13 and ADAMTS4 in the supernatant of harvested OA models were quantified via ELISA (FIG. 17). Data was presented as a mean of all three donors. MMP13 levels were increased following cytokine stimulation (FIG. 17A) as were levels of ADAMTS4 (FIG. 17B).

1.2.4 the Overall Activity of MMPs within the Co-Culture Supernatants

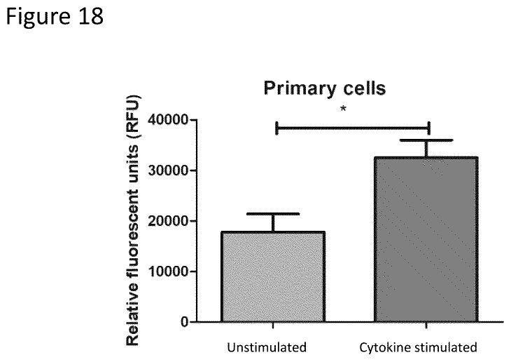

The overall activity of MMPs within the co-culture supernatants were determined using a fluorescence resonance energy transfer assay (FRET), this data is presented in FIG. 18. MMP activity was presented as mean values from three donors. When stimulated with cytokines a statistically significant (P=0.026) increase in the level of MMP activity was recorded, indicating a positive response to cytokine stimuli.

### 1.3 Summary

Primary MSC based OA models were constructed following phenotypic validation via flow cytometry. The OA phenotype of primary cell-based models are comparable to Y201 cell-based models. This data shows that primary MSCs can be used within the OA system in lieu of Y201 MSCs. Additionally, upregulation of MMPs and ADAMTSs were observed at gene expression and protein level in primary cell-based OA models.

### Example 4—Further Improved Model and OA Model

### 1. Optimisation

### 1.1 Method

To improve the overall quality of the model and encourage aggrecan deposition a range of separate coatings were applied to the 3D scaffold. The applied coatings were selected from human collagen type II, human hyaluronan and human aggrecan to empty 3D scaffolds. Specifically, COL-II was solubilised to 1 mg/mL in 0.5M acetic acid. Stock was diluted 1:500 in PBS and 10 uL layered onto each scaffold. Scaffolds were left to air dry overnight at 2-8 degrees. The next day scaffolds there gently washed in PBS and were ready for use. Hyaluronan was reconstituted into a 1% w/v stock in sterile water. 10 uL was added to each scaffold and left to polymerise at 37 degrees for 1 hour. Following this incubation scaffolds were ready to use. Aggrecan was reconstituted in PBS to create a 100ug/mL stock solution. This stock was used neat, with 10 uL added to each scaffold. Scaffolds were incubated at 37 degrees for several hours prior to use.

An independent modification was also made to the chondrogenic media. This involved the addition of the HAPLN1 link protein (SEQ ID NO: 1). Specifically, HAPLN1 stock solution was added to chondrogenic media at a working concentration of 100 ng/mL. The resulting medium composition (complete) is outlined in Table 1. L-Ascorbic acid-2-phosphate and HAPLN1 link protein have limited shelf life at 4° C. and so are added to aliquots of incomplete CDM (all components of Table 1 with the exception of L-Ascorbic acid-2-phosphate and HAPLN1 link protein) to create complete CDM on day 7. Preparation of HAPLN1 link protein (Cat #2608-HP RnD sytems) is as follows:
Unit concentration: 25 μg
Solvent: PBS
Reconstitution volume: 250 μl
Stock concentration: 100 μg/mL
Working concentration: 100 ng/mL
Use: 1:1000 dilution for working concentration. E.g. 50 μL of stock into 50 mL of media.
HAPLN1 is reportedly stable for 1 month at 4° C.

TABLE 1
Improved complete chondrogenic differentiation medium
with the inclusion of HAPLN1 link protein.

| | | | Final | |
|---|---|---|---|---|
| Component | Volume | | Concentration | |
| DMEM High Glucose (4.5 g/l glucose) | 500 | mL | NA | |
| L-glutamine | 5 | mL | 1% | |
| Penicillin- Streptomycin | 5 | mL | 1% | |
| Amphotericin B | 5 | mL | 1% | |
| Dexamethasone | 25 | μL | 50 | nM |
| L-Proline | 500 | μL | 40 | μg/mL |
| ITS | 50 | μL | 1% | (0.01X) |
| TGF-β3 | 25 | μl | 10 | ng/mL |

| | | | |
|---|---|---|---|
| L-Ascorbic acid-2-phosphate | N/A | 50 | μg/mL |
| HAPLN1 link protein | N/A | 100 | ng/mL |

Models were constructed as follows:

1.1.2 Day 0: Seeding MSCs into 3D

Dissociate Y201 MSCs via trypsin, pellet at 2500×rpm then count. Once counted, pellet cells again. Prime Alvetex with 70% ethanol then 3×PBS wash. Transfer scaffolds to a 96-well plate. Suspend cells at a concentration of 25×10⁶/mL and pipette 20 μL of cell suspension directly onto each scaffold. Incubate scaffolds at 37° C. for 3 hours then carefully transfer scaffolds to masterblock 96-well plates. Top up each well with 640 μL of medium and return culture at 37° C. Feed model with 640 μL of DMEM with 10% FBS on day 3 of culture. Maintain cells in this medium (i.e. in DMEM with 10% FBS) for 7 days. This step allows the MSCs to populate the scaffold.

### 1.1.3 Day 7: Chondrogenic Differentiation of MSCs in 3D

At day 7 of culture, carefully remove scaffolds from wells and aspirate DMEM with 10% FBS. Add 640 μL of complete CDM (composition shown in Table 1) to each well, return the scaffolds to the wells and continue to culture at 37° C. Feed cells with 640 mL of complete CDM at least twice weekly up until day 28 of the process. At day 28 the MSCs are fully differentiated chondrocytes and are ready for use. Chondrocytes may be maintained in complete CDM for another 7 days if necessary.

### 1.1.4 Preparation of OA Models

The resulting models were stimulated with cytokines using previously established protocols to create OA models. Following cytokine stimulation, the resulting OA models were harvested and analysed for q-PCR, and immunofluorescent (IF). The supernatant was also harvested and analysed using ELISAs and FRET assays. All analyses were conducted in triplicate.

### 1.2 Results

### 1.2.1 Gene Expression Analysis of Models

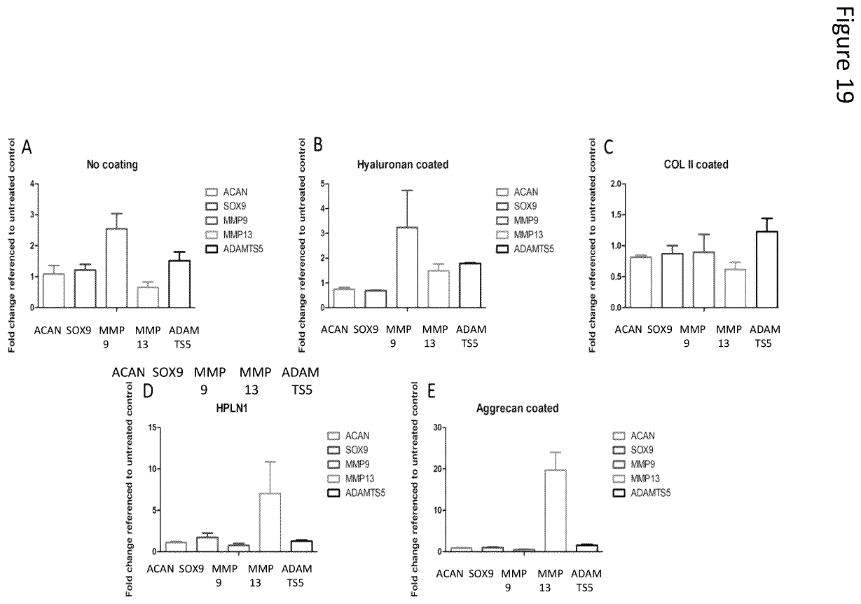

Gene expression analysis of OA models are presented in FIG. 19. The gene expression profile of uncoated OA models is displayed in FIG. 19A. This response was comparable to OA models coated with Hyaluronan (FIG. 19B). Collagen type II coated scaffolds (FIG. 19C) saw an increase in expression of ADAMTS5 much like the uncoated OA models. Both the HAPLN1 supplemented and aggrecan coated OA models saw large increases in fold change of MMP13. The fold changes were in the region of 6-fold and 19-fold respectively.

### 1.2.2 Qualitatively Assessing the Production of Aggrecan

Production of aggrecan was qualitatively assessed using IHC (FIG. 20). Non-coated models, collagen type II coated models and hyaluronan coated models did not stain positively of aggrecan. HAPLN1 supplemented models did stain for aggrecan when models were not stimulated with cytokines. However, HAPLN1 OA models displayed no staining of aggrecan, indicating that there was a loss of aggrecan related to the cytokine response. Unstimulated aggrecan coated models did not stain for aggrecan whereas aggrecan was detected in OA models. This was attributed to issues with the human aggrecan coating rather the model itself. Production of aggrecan by chondrocytes is indicative of correct differentiation of MSCs to chondrocytes.

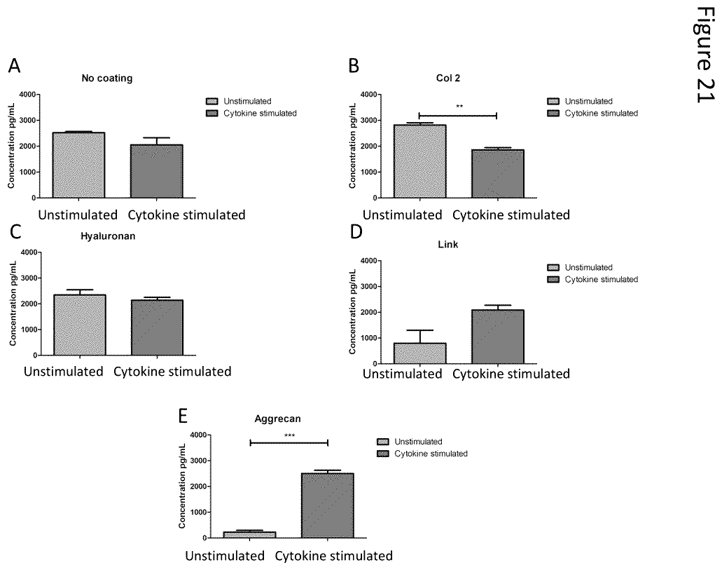

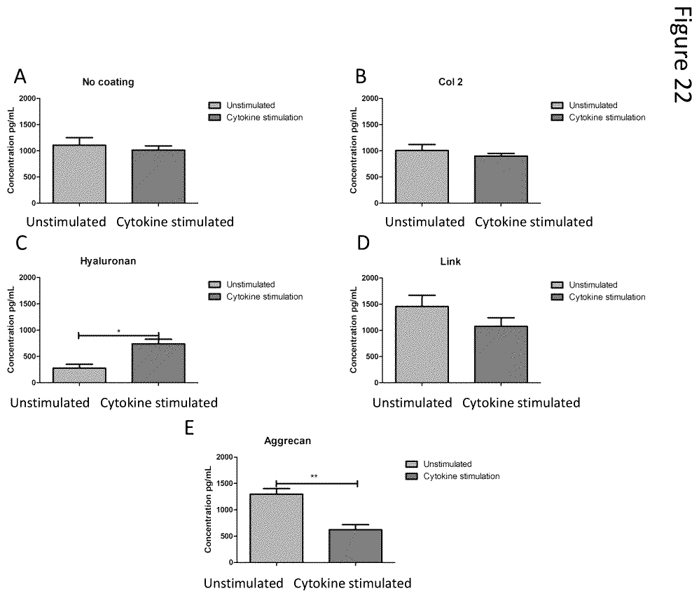

1.2.3 Quantitively Assessing the Concentration of MMP13 and ADATS4 Levels The concentration of MMP13 present within the cell culture supernatant was quantified via ELISA (FIG. 21). OA models which were not coated (FIG. 21A), coated in collagen II (FIG. 21B), and coated in hyaluronan (FIG. 21C) showed a decrease in the levels of MMP13. However, HAPLN1 supplemented models (FIG. 21D) and aggrecan coated OA models (FIG. 21E) resulted in increased MMP13 levels, particularly aggrecan coated models which resulted in statistically significant increases. Quantification of ADAMTS4 via ELISA is displayed in FIG. 22. Hyaluronan coated models resulted in an increase in ADATS4 levels (FIG. 22C).

### 1.2.4 Activity of MMP Molecules Generated by the Models Versus OA Models

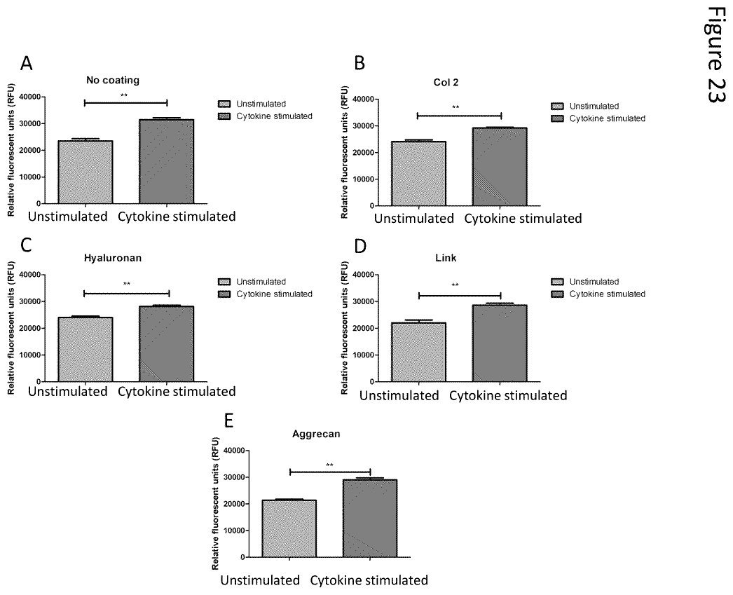

The activity of MMP molecules generated by the OA models were determined via FRET assay (FIG. 23). Overall, all OA models showed a statistically significant increase in MMP activity.

### 1.3 Summary

The use of HAPLN1 in the chondrogenic differentiation medium has improved the physiological relevance of the overall model by encouraging chondrocytes to produce aggrecan, a protein which is normally also produced by chondrocytes in vivo. Y201 chondrocytes supplemented with HAPLN1 were shown to produce human aggrecan which appeared to diminish following cytokine stimulation. Furthermore, adding HAPLN1 protein to the chondrogenic differentiation medium, human aggrecan can be synthesised by the cells within the 3D scaffold, a crucial step in improving physiological relevance of the osteochondral construct and improving the quality of both the model and OA model (after cytokine stimulation). Cytokine stimulation of HAPLN1 models resulted in loss of aggrecan matrix according to IHC data. This is currently a novel development which has yet to be seen in the literature.

### Discussion of Examples 2-4

The initial characterisation of MMP activity (FIG. 13) indicated that the OA model could generate a tissue degradation phenotype with statistically significant change. The addition of the small molecule mixture inhibitor to the OA model demonstrated that the degradation activity could be modified through a potential therapeutic. The RNA seq analysis further validated historical data indicating that the OA model elevated MMP levels in response to cytokine treatment.

Preliminary use of primary MSCs within the model and resulting OA model as opposed to immortalised Y201s indicated the phenotypic response of the cells to cytokine stimulation was similar regardless of the source of MSCs used. Primary MSC based OA models produced an increase in degradation proteins such as MMP13 and ADAMTS4 (FIG. 17) like historical data of Y201 based models (data not shown). Additionally, MMP activity of primary cell-based models also showed significant increases, which were also comparable to previous data generated form Y201 based models.

The improvements made to the OA system through modification of chondrogenic media resulted in the synthesis of aggrecan within the 3D scaffold (FIG. 24). This network of aggrecan was constant throughout the scaffold and repeated across all three experimental triplicates (data not shown). Aggrecan was synthesised to a far greater extent than any historical experiments had demonstrated, indicating that the development of the OA model was significantly improved by use of the HAPLN1 protein during chondrogenic differentiation. Following cytokine stimulation, the aggrecan matrix was observed to be diminished. This is a significant observation as the data implied that the OA phenotype as a result of cytokine stimulation resulted in degradation of the aggrecan present within the system.

### REFERENCES

Sally James, James Fox, Farinaz Afsari, Jennifer Lee, Sally Clough, Charlotte Knight, James Ashmore, Peter Ashton, Olivier Preham, Martin Hoogduijn, Raquel De Almeida Rocha Ponzoni, Y. Hancock, Mark Coles, Paul Genever, (Multiparameter Analysis of Human Bone Marrow Stromal Cells Identifies Distinct Immunomodulatory and Differentiation-Competent Subtypes) Stem Cell Reports. 2015 Jun. 9; 4(6): 1004-1015. PMCID: PMC4471830

Amino acid sequence of human HAPLN1
SEQ ID NO: 1
MKSLLLLVLISICWADHLSDNYTLDHDRAIHIQAENGPHLLVEAEQA
KVFSHRGGNVTLPCKFYRDPTAFGSGIHKIRIKWTKLTSDYLKEVDV
FVSMGYHKKTYGGYQGRVFLKGGSDSDASLVITDLTLEDYGRYKCEV
IEGLEDDTVVVALDLQGVVFPYFPRLGRYNLNFHEAQQACLDQDAVI
ASFDQLYDAWRGGLDWCNAGWLSDGSVQYPITKPREPCGGQNTVPGV
RNYGFWDKDKSRYDVFCFTSNFNGRFYYLIHPTKLTYDEAVQACLND
GAQIAKVGQIFAAWKILGYDRCDAGWLADGSVRYPISRPRRRCSPTE
AAVRFVGFPDKKHKLYGVYCFRAYN

### EMBODIMENTS

1. A cellular composite comprising a 3D (three dimensional) cell growth material within which a population of chondrocytes is distributed, and which has a surface that is coated with a population of osteoblasts.

2. The cellular composite according to embodiment 1, wherein the population of chondrocytes has normal expression levels of a protein selected from the group consisting of aggrecan, SOX9, ADAMTS5, MMP9 and MMP13, optionally wherein the normal expression levels are substantially the same as expression levels in healthy chondrocytes.

3. The cellular composite according to embodiment 1, wherein the population of chondrocytes has abnormal expression levels of a protein selected from the group consisting of aggrecan, SOX9, ADAMTS5, MMP9 and MMP13, optionally wherein the abnormal expression levels in chondrocytes are substantially the same as expression levels in chondrocytes from a subject having osteoarthritis and/or expression levels in chondrocytes that have been exposed to osteoarthritic inducing cytokines, further optionally wherein the cytokines are selected from the group consisting of IL1-α and/or oncostatin M.

4. The cellular composite according to any preceding embodiment, wherein the chondrocytes and/or osteoblasts are derived from mesenchymal stem cells (MSCs), optionally wherein the MSCs are Y201 cells.

5. The cellular composite according to any preceding embodiment, wherein the chondrocytes and/or osteoblasts are human cells.

6. The cellular composite according to any preceding embodiment, wherein the 3D cell growth material is formed from a scaffold or a gel.

7. The cellular composite according to embodiment 6, wherein the scaffold comprises or consists of a polymer, optionally wherein the polymer is selected from the group consisting of polystyrene, Teflon®, polycarbonate, polyester, or acrylate, further optionally wherein the scaffold is Alvetex®.

8. The cellular composite according to embodiment 6, wherein the gel is a hydrogel, optionally wherein the hydrogel is selected from the group consisting of HydroMatrix™ Peptide Hydrogel, MaxGel™ Human ECM, Hystem® Stem Cell Culture, Geltrex®, or Matrigel™.

9. The cellular composite according to any preceding embodiment, wherein the pores are between about 25-500 μm, or between about 100-300 μm, or between about 150-250 μm in size.

10. The cellular composite according to any preceding embodiment, comprising a further population of cells, optionally wherein the further population of cells comprises or consists of neurons and/or Schwann cells.

11. A method of producing a cell composite comprising:
a) distributing a population of chondrocytes within a 3D cell growth material; and
b) coating a surface of the 3D cell growth material with a population of osteoblasts.

12. The method according to embodiment 11, wherein the step of distributing a population of chondrocytes comprises distributing a population of mesenchymal stem cells (MSCs) within the 3D cell growth material and differentiating the MSCs into chondrocytes.

13. The method according to embodiments 11 or 12, wherein the method further comprises contacting the population of chondrocytes with IL1-α and/or oncostatin M, optionally wherein the for 1 day, about 2 days, about 3 days, about 4 days, about 5 days, about 6 days, about 7 days, about 8 days, about 9 days, about 10 days.

14. The method according to any one of embodiments 11 to 13, wherein the chondrocytes and/or osteoblasts are derived from MSCs, optionally wherein the MSCs are Y201 cells.

15. The method according to any one of embodiments 11 to 14, wherein the cells are human cells.

16. The method according to any one of embodiments 11 to 15, wherein the 3D cell growth material is formed from a porous scaffold and/or a gel.

17. The method according to embodiment 16, wherein the scaffold comprises or consists of a polymer, optionally wherein the polymer is selected from the group consisting of polystyrene, Teflon®, polycarbonate, polyester, or acrylate, further optionally wherein the scaffold is Alvetex®.

18. The method according to embodiment 16 or 17, wherein the gel is a hydrogel, optionally wherein the hydrogel is selected from the group consisting of HydroMatrix™ Peptide Hydrogel, MaxGel™ Human ECM, Hystem® Stem Cell Culture, Geltrex®, or Matrigel™.

19. The method according to any one of embodiments 11 to 18, wherein the pores are between about 25-500 μm, or between about 100-300 μm, or between about 150-250 μm in size.

20. The method according to any one of embodiments 11 to 19, comprising providing a further population of cells, optionally wherein the further population of cells comprises or consists of neurons and/or Schwann cells.

21. A cell composite produced by a method according to any one of embodiments 11 to 20.

22. An in vitro model for studying the physiology or pathophysiology of articular cartilage comprising the composite according to any one of embodiments 1 to 10, or 21.

23. The model according to embodiment 22, wherein the pathophysiology is osteoarthritis and/or osteoarthrosis.

24. Use of a cellular composite according to embodiments 1 to 10 or 21 as an in vitro model of articular cartilage.

25. The use of embodiment 24, wherein the articular cartilage is heathy or diseased.

26. The use of embodiment 25, wherein the disease is osteoarthritis and/or osteoarthrosis.

27. A method of screening an agent for the treatment or prevention of articular cartilage disease, comprising:
a) providing a composite according to claims 1 to 10 or 21;
b) exposing the composite to the agent; and
c) determining whether the agent has a therapeutic effect on the composite.

28. The method according to embodiment 27, wherein the articular cartilage disease is osteoarthritis and/or osteoarthrosis.

29. The method according to embodiment 27 or 28, wherein when the agent is for treatment, the population of chondrocytes has abnormal expression levels of a protein selected from the group consisting of aggrecan, SOX9, ADAMTS5, MMP9 and MMP13, or wherein when the agent is for prevention, the population of chondrocytes has normal expression levels of a protein selected from the group consisting of aggrecan, SOX9, ADAMTS5, MMP9 and MMP13.

30. The method according to any one of embodiments 11 to 20, wherein the 3D cell growth material has been coated with aggrecan prior to step a).

1. A method of producing a cellular composite comprising:
a) distributing a population of mesenchymal stem cells (MSCs) within a three-dimensional (3D) cell growth material and differentiating the MSCs into chondrocytes in a cell culture medium supplemented with a protein of the hyaluronan and proteoglycan binding link protein (HAPLN) family; and
b) coating a surface of the 3D cell growth material with a population of osteoblasts.

2. The method of claim 1, wherein the surface of the 3D cell growth material with the population of osteoblasts is coated after MSCs have been differentiated into chondrocytes.

3. The method of claim 1, wherein the cell culture medium is supplemented with the protein of the HAPLN family at a concentration of about 50 ng/ml to about 150 ng/ml.

4. The method of claim 1, wherein the protein of the HAPLN family is HAPLN1.

5. (canceled)

6. The method of claim 1, wherein the cell culture medium is supplemented with the protein of the HAPLN family from about day 0 to about day 28 of the differentiation step.

7. The method of claim 1, wherein the method further comprises contacting the population of chondrocytes with IL1-α and/or oncostatin M after step b).

8. The method of claim 7, wherein the population of chondrocytes is contacted with IL1-α and/or oncostatin M for about 1 day to about 10 days.

9. The method of claim 1, wherein the chondrocytes and/or osteoblasts are derived from MSCs.

10. (canceled)

11. The method of claim 1, wherein the 3D cell growth material is formed from a porous scaffold and/or a gel.

12. The method of claim 11, wherein the porous scaffold comprises a polymer.

13. The method of claim 12, wherein the gel is a hydrogel.

14. The method of claim 11, wherein pores of the porous scaffold are between about 25-500 μm in size.

15. The method of claim 1, further comprising providing a further population of cells.

16. A cellular composite produced by a method of claim 1.

17. An in vitro model for studying the physiology or pathophysiology of articular cartilage comprising the composite according to claim 16.

18. The model of claim 17, wherein the pathophysiology is selected from the group consisting of osteoarthritis, osteoarthrosis and rheumatoid arthritis.

19. (canceled)

20. (canceled)

21. (canceled)

22. A method of screening an agent for the treatment or prevention of articular cartilage disease, comprising:
a) providing a composite according to claim 16;
b) exposing the composite to the agent; and
c) determining whether the agent has a therapeutic effect on the composite.

23. The method of claim 22, wherein the articular cartilage disease is selected from the group consisting of osteoarthritis, osteoarthrosis and rheumatoid arthritis.

24. The method of claim 22, wherein when the agent is for treatment, the population of chondrocytes has abnormal expression levels of a protein selected from the group consisting of aggrecan, SOX9, ADAMTS5, MMP9 and MMP13.

25. The method of claim 22, wherein when the agent is for prevention, the population of chondrocytes has normal expression levels of a protein selected from the group consisting of aggrecan, SOX9, ADAMTS5, MMP9 and MMP13.
